(19) United States
(12) Patent Application Publication
(10) Pub. No.: US 2011/0190662 A1
(43) Pub. Date: Aug. 4, 2011

(54) RAPID EXCHANGE FNA BIOPSY DEVICE WITH DIAGNOSTIC AND THERAPEUTIC CAPABILITIES

(75) Inventor: John McWeeney, Brighton, MA (US)

(73) Assignee: BEACON ENDOSCOPIC CORPORATION, Danvers, MA (US)

(21) Appl. No.: 13/029,593

(22) Filed: Feb. 17, 2011

Related U.S. Application Data
(63) Continuation-in-part of application No. 12/243,367, filed on Oct. 1, 2008.
Application No. 12/243,367 is a continuation-in-part of application No. 12/607,636, filed on Oct. 28, 2009.
(60) Provisional application No. 61/117,966, filed on Nov. 26, 2008.
(60) Provisional application No. 61/152,741, filed on Feb. 16, 2009.
(60) Provisional application No. 61/305,304, filed on Feb. 17, 2010.
(60) Provisional application No. 61/305,396, filed on Feb. 17, 2010.

(51) Int. Cl. A61B 10/02

(52) U.S. Cl. 600/567

(57) ABSTRACT

A device for needle biopsy and delivery of a diagnostic or therapeutic agent is presented. The device includes a handle member having proximal and distal portions. A proximal handle member is disposed to the proximal portion of the handle member and a distal handle member is disposed to the distal portion of the handle member. A sheath lumen is disposed within the handle member and extends from the distal portion of the handle member. A needle housing member is partially disposed to the proximal portion of the handle member and a needle is disposed within the sheath lumen. The needle housing member includes one or more ports for introducing an agent or a device into the housing member. The needle can include an agent or a device disposed at a distal portion thereof.

### CROSS REFERENCE TO RELATED APPLICATIONS

This application is a continuation-in-part application of U.S. patent application Ser. No. 12/243,367, filed on Oct. 1, 2008, and a continuation-in-part of U.S. patent application Ser. No. 12/607,636, filed on Oct. 28, 2009, which in turn claims the benefit of U.S. Provisional Application Ser. No. 61/117,966, filed on Nov. 26, 2008, and U.S. Provisional Application Ser. No. 61/152,741 filed on Feb. 16, 2009. The contents of each of these applications are incorporated by reference herein in their entireties. This application further claims priority under 35 U.S.C. 119(e) to U.S. Provisional Application No. 61/305,304, filed on Feb. 17, 2010, and U.S. Provisional Application No. 61/305,396, filed on Feb. 17, 2010, the contents of which are incorporated by reference herein in their entireties.

### FIELD OF THE INVENTION

The present invention generally relates to biopsy devices, and more particularly, to needle biopsy devices for collecting tissue, fluid and cell samples in conjunction with procedures such as endoscopic ultrasound or endoscopic bronchial ultrasound. The devices of the invention are further configured for delivering a diagnostic or therapeutic agent to a targeted tissue site.

### BACKGROUND OF THE INVENTION

Endoscopic ultrasound procedures have been used for more than twenty five years within the field of medicine. These procedures allow clinicians to scan, locate and identify individual layers of a patient's gastrointestinal tract to determine the location of individual mucosal and sub-mucosal layers. Once identified, appropriate therapeutic modes of treatment for malignancies and various abnormalities may be determined by a clinician.

An endoscopic ultrasound procedure may consist of several steps. For example, a clinician may sedate a patient and insert a probe via esophagogastroduodenoscopy into the patient's stomach and duodenum. An endoscope may then be passed through the patient's mouth and advanced to the level of the duodenum. From various positions between the esophagus and duodenum, organs or masses outside the gastrointestinal tract may be imaged to determine abnormalities. If any abnormalities are present, the organs or masses can be biopsied through fine needle aspiration. Organs such as the liver, pancreas and adrenal glands are easily biopsied as are any abnormal lymph nodes. A patient's gastrointestinal wall can also be imaged to determine the presence of any abnormalities. For example, abnormal thickness within a patient's gastrointestinal wall may be suggestive of inflammation or malignancy.

The quality of images produced via endoscopic ultrasounds is directly proportional to the level of frequency used. Although a high frequency ultrasound can produce a higher image quality, high frequency ultrasounds do not penetrate organ walls as well as lower frequency ultrasound. As a result, the examination of the nearby organs is not possible.

Mediastinoscopy is a prevailing method for determining the presence of nodal metastases in the mediastinum. Generally performed as an outpatient surgical procedure, mediastinoscopy is associated with a low rate of serious adverse effects and is considered to be highly accurate. Endobronchial ultrasound guided fine needle aspiration biopsy of mediastinal nodes offers a less invasive alternative for histologic sampling of the mediastinal nodes. Endobronchial ultrasound has been widely adopted by pulmonologists and is poised to replace mediastinoscopy in the future. For thoracic surgeons, endobronchial ultrasound can be easily learned and it may be important to do so if their specialty is to maintain the traditional and important role in the diagnosis and staging of thoracic malignancies.

During endobronchial ultrasound, a clinician can perform needle aspiration on lymph nodes using a bronchoscope inserted through the mouth. For an endobronchial ultrasound procedure, an endoscope fitted with an ultrasound processor and a fine-gauge aspiration needle is guided through a patient's trachea. Once appropriately positioned, the needle portion of the fine needle aspiration device is advanced into the lymph node, the sample aspirated, and device is removed from the bronchoscope.

Endoscopic ultrasounds and endoscopic bronchial ultrasounds through fine needle aspiration are presently standard modes of diagnosis in the field of gastrointestinal endoscopy and bronchoscopy. These procedures traditionally result in high yields of sensitivity and specificity in the diagnosis and management of indications of diseases such as esophageal cancer, pancreatic cancer, liver mass, non-small cell lung cancer, pancreatic mass, endobronchial mass, and intra-abdominal lymph nodes.

An endoscopic ultrasound through fine needle aspiration requires a fine needle aspiration device that is attached to the luer port or working channel of a typical echoendoscope. Traditional devices utilize a series of push and pull handles to control the axial movement of the catheter shaft of the device and the depth of needle penetration. These device, however, suffer from several drawbacks.

For example, the means of attaching a device to an echoendoscope is cumbersome. Devices presently utilize male fitting adapters that must be screwed onto a female luer port of an endoscope. In addition, these devices provide sub-optimal ergonomics of use. More specifically, a clinician must actuate a number of handles independently and lock respective handles in position via cap screw arrangement to secure the device. The cumulative actions required by a clinician result in significantly drawn out procedures. Further, needles commonly kink or deform during removal from a device causing numerous delays and failures. Moreover, multiple passes per procedure are required, which prolong the procedure and result in a clinician needing to reconfirm the location of a needle relative to a desired aspiration site with each new pass.

Needles are commonly used in medical procedures, with biopsy being a primary field of use for such devices. However, in the current field of therapeutic endoscopy and pulmonology, the ability of the physician to effectively and efficiently treat known metastases, strictures, tumors etc., in the gastrointestinal tract, the endobronchial tract, and peripheral areas, is greatly hampered by the lack of available technology to deliver diagnostic aids, reagents and other therapeutic means to the desired treatment site.

Therefore, a need exists for improved devices for delivering diagnostic aids, reagents and/or therapeutic means to a desired treatment site for use in endoscopic ultrasound procedures.

### SUMMARY OF THE INVENTION

The present invention provides needle biopsy devices, particularly fine needle aspiration (FNA) devices, and methods for collecting tissue, fluid, cell samples from the body in conjunction with an Endoscopic Ultrasound (EUS) or Endoscopic Bronchial Ultrasound (EBUS) procedure. The devices of the present invention are further configured for use as a conduit or delivery system to deliver therapies to a desired site in the human gastrointestinal and respiratory systems.

The devices of the invention are modular in that the needle housing member detaches from the proximal handle of the device for each individual “pass” or aspirated sample taken by the endoscopist at the site of the lesion or abnormality.

In one embodiment, the FNA device of the invention includes a handle member having proximal and distal portions. A proximal handle member is disposed to the proximal portion of the handle member, and a distal handle member is disposed to the distal portion of the handle member. A sheath lumen is disposed within the handle member and extends from the distal portion of the handle member. A needle housing member is partially disposed in the proximal handle member and includes at least two ports for introducing a device or agent into the housing member. One port may be used to insert a device, such as a stylet, into the needle housing member. The second port can be used to introduce an agent, such as a therapeutic agent (in liquid, gel or glue form), or a diagnostic agent (e.g., an imaging agent), into the needle housing member. Alternatively, the second port can be used to introduce a device, such as a syringe or fiber optic probe into the needle housing member. The needle housing member is moveable in a substantially transverse direction relative to the longitudinal axis of the handle member. The FNA device further includes a needle disposed within the sheath lumen.

In an alternate embodiment, the FNA devices of the invention include a handle member having proximal and distal portions. A proximal handle member is disposed to the proximal portion of the handle member, and a distal handle member is disposed to the distal portion of the handle member. A sheath lumen is disposed within the handle member and extends from the distal portion of the handle member. A needle housing member is partially disposed in the proximal handle member and is moveable in a substantially transverse direction relative to the longitudinal axis of the handle member. A needle is disposed within the sheath lumen. The needle includes an agent or a device disposed in or on a distal portion of the needle.

As will be described later, the devices of the invention can be configured to deliver agents in the form of liquids, gels, glues, seeds, pellets, encapsulated liquids or encapsulated gels, to desired locations in the gastrointestinal and respiratory systems to treat various cancerous or other tumors. For example, the devices of the invention can be used to facilitate the efficient delivery of a desired agent, such as chemotherapeutic agents, sclerosing agents, tumor necrosing factors, growth factors (pharma and bio agents), and/or imaging agents to desired sites in the gastrointestinal or respiratory tracts, to provide a localized therapeutic effect in the treatment of benign and malignant tumors. The devices of the invention can also be used to deliver radiation therapy directly to tumors diagnosed in the respiratory and gastrointestinal tracts to aid in the treatment of cancer. The desired agent can be delivered to the target site by introducing the desired agent in a liquid, gel or glue form through one or more of the ports in the needle housing member. Alternatively, the desired agent can be delivered to the target site using a needle pre-loaded with the desired agent in an encapsulated form, or pellet or seed form.

The devices of the invention can also be used to facilitate the efficient delivery of fiducial markers to desired sites in the gastrointestinal or respiratory tracts, to provide “landmarks” in the anatomy to aid in directed radiotherapy or other therapy.

The devices of the invention can also be used to facilitate the efficient delivery of implantable biomarkers that may be used as part of an image guided system in conjunction with Fine Needle Aspiration (FNA) using ultrasound guided imaging techniques to desired sites in the gastrointestinal or respiratory tracts, to provide an identifying position for the delivery of subsequent therapy during patient treatment.

The devices of the invention can also be used to deliver neuromodulation/pacing leads to specific areas in the gastrointestinal (GI) and respiratory tracts as well as other areas of the human anatomy.

The devices of the invention can also be used to facilitate the delivery of a metal or polymeric self expanding or laser cut stent which may be used as a conduit to provide drainage in the gastrointestinal system between the following anatomical entities, including but not limited to: the gall bladder and the left hepatic and stomach, gall bladder drainage from the gall bladder to the duodenum, gall bladder drainage from the gall bladder to stomach, the pancreas to the stomach and uncinate of the duodenum, and pseudocyst drainage into the stomach.

As with the delivery of an agent in an encapsulated form, or a pellet or seed form, one or more of a desired fiducial marker, biomarker and/or stent can be delivered to the target site using a needle pre-loaded with the desired fiducial marker, biomarker and/or stent.

The devices of the invention can also be used to facilitate the delivery of optical imaging capability (e.g., imaging agents and/or devices such as fiber optic imaging) to areas in the gastrointestinal (GI) and respiratory tracts as well as the abdominal cavity.

In yet another embodiment, the FNA devices of the invention provide the end user with real-time validation of the aspirated cellular sample. For example, the FNA device includes a handle member having proximal and distal portions. A proximal handle member is disposed to the proximal portion of the handle member, and a distal handle member is disposed to the distal portion of the handle member. A sheath lumen is disposed within the handle member and extends from the distal portion of the handle member. A needle housing member is partially disposed in the proximal handle member and includes an indicator assay insert mounted to an inner wall in a proximal portion of the needle housing member. The needle housing member further includes a clear window in the proximal portion of the needle housing member, preferably mounted opposite the indicator assay insert. The device further includes a needle disposed within the sheath lumen.

In each of the aforementioned embodiments, the FNA devices of the invention can further include a strain relief to stabilize the needle by providing a smooth transition and bend radius for the needle housing member upon insertion and removal from the proximal handle member.

The various embodiments of the FNA devices described herein can further include a connecting member. The connecting member has at least one indentation for engaging to at least one adaptation member in the proximal handle member of the device. In certain embodiments, the proximal handle member includes a release member for engaging and disengaging the needle housing member.

Other features and advantages of the invention will be apparent from the following detailed description and claims.

### BRIEF DESCRIPTION OF THE DRAWINGS

In the drawings, like structures are referred to by like numerals throughout the several views. Note that the illustrations in the figures are representative only, and are not drawn to scale, the emphasis having instead been generally placed upon illustrating the principles of the invention and the disclosed embodiments. In the following description, various embodiments of the present invention are described with reference to the following drawings.

FIG. 1 is a perspective view of a needle biopsy device.

FIG. 2 is a perspective view of a handle member.

FIG. 3 is a cross-sectional view of a proximal portion of a handle member.

FIG. 4 is a cross-sectional view of a proximal portion of a handle member and a proximal handle member.

FIG. 5 is a cross-sectional view of an assembled proximal portion of a needle biopsy device.

FIG. 6 is a partial cross-sectional view of an assembled distal portion of a needle biopsy device.

FIG. 7 is a perspective view of an assembled distal portion of a needle biopsy device.

FIG. 8 is a cross-sectional view of a connector according to another embodiment of the invention.

FIG. 9 is a perspective view of a connector according to another embodiment of the invention.

FIG. 10 is a cross-sectional view of a connector according to another embodiment of the invention.

FIG. 11 is a partial cross-sectional view of a disassembled distal portion of the invention.

FIG. 12 is a perspective view of a needle housing member.

FIG. 13 is a perspective view of a needle housing member according to another embodiment of the invention.

FIG. 14 is a perspective view of a needle housing member according to another embodiment of the invention.

FIG. 15 is a perspective view of a needle housing member according to another embodiment of the invention.

FIG. 16 is a cross-sectional view of a needle housing member according to another embodiment of the invention.

FIG. 17 is a drawing depicting an alternate embodiment for the needle housing member with a bifurcated hub detail.

FIG. 18 is a cross-sectional drawing of the bifurcated needle hub member.

FIG. 19 is a cross sectional drawing depicting a needle embodiment with pre-loaded liquid media therapy in the needle component of the needle housing member.

FIG. 20 is an alternate embodiment of a needle housing member with pre-loaded therapy.

FIG. 21 is a drawing depicting an alternate embodiment for a needle housing member with pre-loaded fiducial markers, biomarkers, and/or radioactive seeds, etc., in the needle component of the needle housing member.

FIG. 22 is drawing depicting an alternate embodiment for a needle housing member with pre-loaded self-expanding stent in the needle component of the needle housing member.

FIG. 23 is a drawing depicting deployment of a self expanding stent from a needle member.

FIG. 24 is a drawing depicting a self-expanding stent post deployment from a needle member.

FIG. 25 is a drawing depicting an alternate embodiment for a needle housing member with pre-loaded self-expanding stent in the needle component of the housing member.

FIG. 26 is a cross sectional drawing depicting a self-expanding stent compressed in a catheter sheath of the present invention.

FIG. 27 is a drawing depicting deployment of a self expanding stent from a needle member.

FIG. 28 is a drawing of the clinical field of use and how the present self expanding stent invention may be used.

FIG. 29 is a drawing of the clinical field of use and how the present self expanding stent invention may be used.

FIG. 30 is a drawing of an alternate design for a self-expanding stent.

FIG. 31 is a drawing of the clinical field of use and how the present self expanding stent invention may be used.

FIG. 32 is an illustration of a typical fiber optic imaging system used in the intended field of use for the invention.

FIG. 33 is a drawing of how the present invention may be used in conjunction with an optical imaging system.

FIG. 34 is a drawing depicting how the present invention may be used in a clinical procedure.

FIG. 35 is alternate embodiment of the present invention to provide real time analysis of an aspirated cellular sample.

FIG. 36 is a cross sectional drawing of an alternate embodiment for the needle housing member hub detail.

### DETAILED DESCRIPTION

The exemplary embodiments of the needle biopsy device and methods of operation disclosed are discussed in terms of needle biopsy devices for collecting tissue, fluid, and cell samples from a body in conjunction with an endoscopic ultrasound or endoscopic bronchial ultrasound. It is envisioned that the present disclosure, however, finds application to a wide variety of biopsy devices for the collection of samples from a subject. It is also envisioned that the present disclosure may be employed for collection of body fluids including those employed during procedures relating to phlebotomy, digestive, intestinal, urinary, veterinary, etc. It is contemplated that the needle biopsy device may be utilized with other needle biopsy applications including, but not limited to, fluid collection, catheters, catheter introducers, spinal and epidural biopsy, aphaeresis, dialysis, etc. Suitable needle biopsy devices are described in U.S. patent application Ser. No. 12/243,367 (published as U.S. Published Patent Application No. US2010/0081965), and U.S. patent application Ser. No. 12/607,636 (published as U.S. Published Patent Application No. US2010/0121218), the contents of which are each incorporated by reference herein in their entireties.

It is even further envisioned that the needle biopsy devices described herein may be utilized for delivering a diagnostic or therapeutic agent to a targeted tissue site.

In the discussion that follows, the term “proximal” refers to a portion of a structure that is closer to a clinician, and the term “distal” refers to a portion that is further from the clinician. According to the present disclosure, the term “clinician” refers to an individual performing sample collection, installing or removing a needle from a needle biopsy device, and may include support personnel. Reference will now be made in detail to exemplary embodiments of the disclosure, which are illustrated in the accompanying figures.

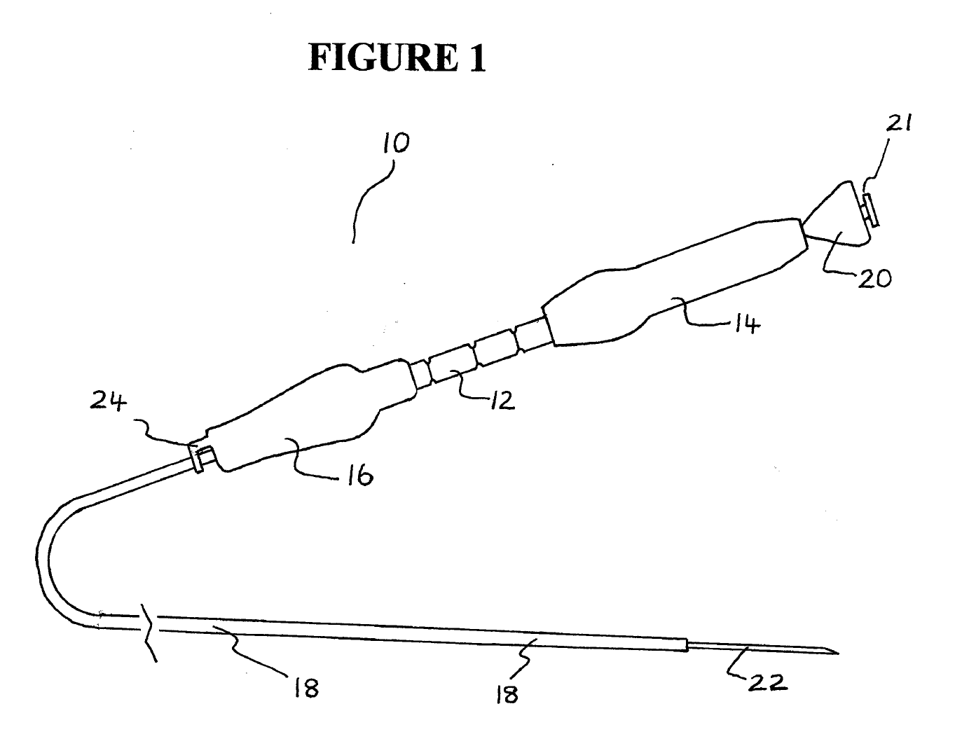

Referring to FIG. 1, a needle biopsy device 10 is provided for fine needle aspiration during procedures such as endoscopic ultrasound. The device 10 is generally comprised of a handle 12, a proximal handle member 14, a distal handle member 16, a sheath lumen 18, a needle housing member 20, a stylet 21, a needle 22, and a connector 24.

In one embodiment, a clinician connects the device 10 to another medical device via the connector 24. The clinician subsequently inserts the needle housing member 20, which includes the stylet 21 and the needle 22, into the proximal portion of the proximal handle member 14. The stylet 21 may be, but is not limited to, a removable coaxial thin wire which is passed within the lumen of the needle 22. It is envisioned that the stylet 21 may provide rigidity and stability to the needle 22. Additionally, it is contemplated that the stylet 21 can protect the needle 22 from damage or inadvertent collection of samples.

Upon passing the needle 22 through the sheath lumen 18, the clinician may slideably manipulate the proximal handle member 14 and the distal handle member 16 along the axis of the handle 12. At this juncture, the clinician may lock the proximal handle member 14 and the distal handle member 16 at various depths along the handle 12. Movement of the proximal handle member 14 causes the needle 22 to extend from the distal portion of the sheath 18. Additionally, movement of the distal handle member 16 adjusts the depth of exposure of the sheath 18. A clinician may subsequently withdraw the stylet 21 from the needle housing member 20 and begin needle aspiration.

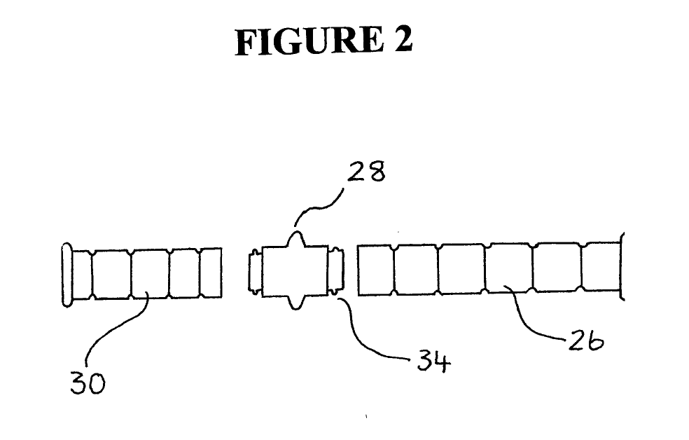

Referring to FIG. 2, the handle 12 includes a proximal portion 26, a distal portion 30, and a stop portion 28. The handle 12 may be monolithically formed and injection molded from a rigid polymer such as acrylonitrile butadiene styrene, polystyrene, polyetherkeytone, polyamide, polyethersulfone, polyurethane, ether block amide copolymers, polyacetal, and derivatives thereof. It is contemplated that the handle 12 can be integrally assembled of multiple sections and may be substantially transparent, opaque, etc. The handle 12 may also be variously configured and dimensioned such as, for example, rectangular, spherical, tapered etc.

The handle 12 can be joined by any appropriate process such as, for example, snap fit, adhesive, solvent weld, thermal weld, ultrasonic weld, screw, rivet, etc. In this configuration, the handle 12 is presented wherein the proximal portion 26, the distal portion 30, and the stop portion 28 are joined through a snap fit process. In one embodiment, the handle 12 is assembled by inserting the stop portion 28 into the proximal portion 26, and subsequently inserting the distal portion 30 into the stop portion 28. The stop portion 28 is disposed between the proximal portion 26 and the distal portion 30 to prevent axial movement of the proximal 14 and distal 16 handle members, as shown in FIG. 1, into one another.

The stop portion 28 takes the form of a circular ring with details 34 that are incorporated into the molding. The details 34 facilitate the insertion of the stop portion 28 into proximal portion 26 and the distal portion 30 of the handle 12. It is envisioned that the details 34 may create a permanent binding between the proximal portion 26, the stop portion 28, and the distal portion 30.

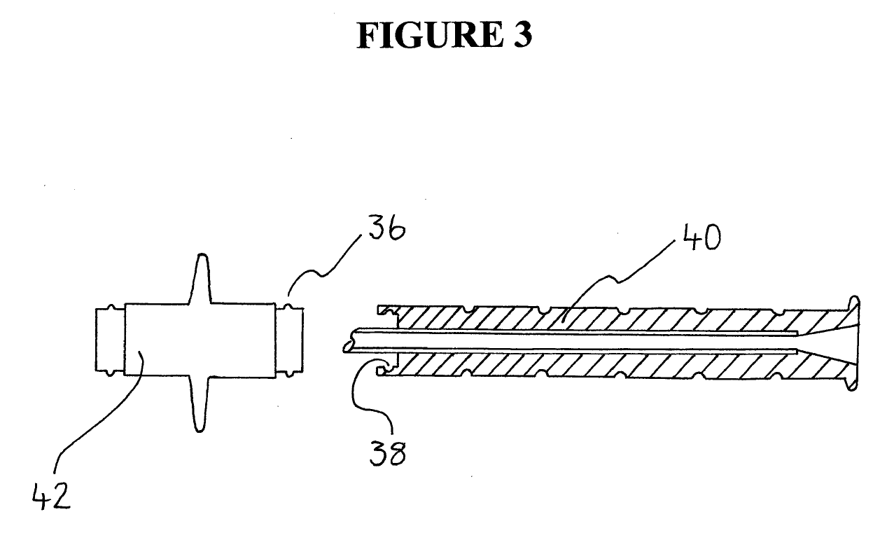

Referring to FIG. 3, an alternative embodiment is presented wherein the details 36 consist of a male and female mating configuration. The details 36 consists of a raised circular male ridge that fits into a female type depression 38 in the proximal portion of a handle 40. It is envisioned that an identical configuration can exist between the details 36 and the distal portion (not shown in Figure) of the handle 40. A configuration is further contemplated wherein a stop portion 42 includes details 36 that are female type depressions and the proximal and distal portions of the handle 40 includes a raised circular male ridge.

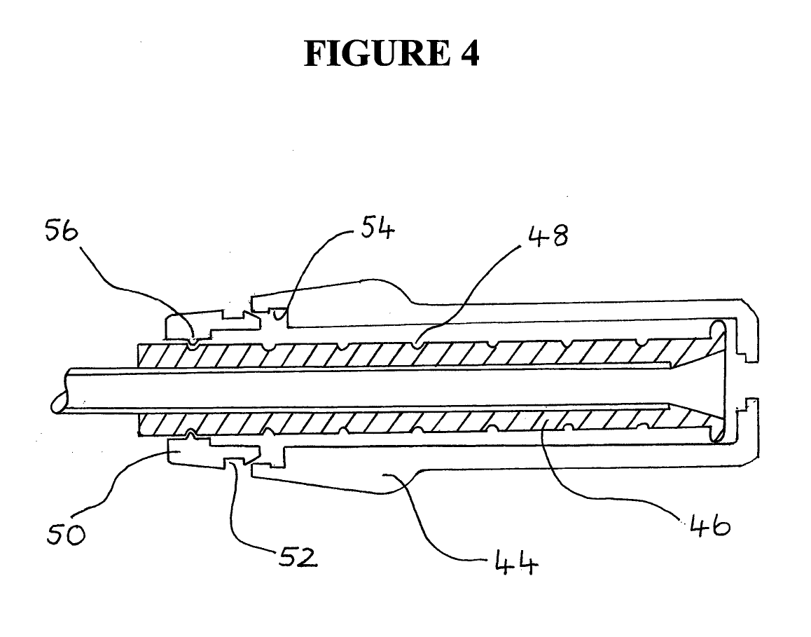

Turning to FIG. 4, a proximal portion of a handle 46 is presented wherein a proximal handle member 44 is disposed thereon. The handle 46 includes indentations 48 to facilitate slideable engagement along the axis of the handle 46. The indentations 48 may take the form of ribs, ridges, or other forms of detents. In a preferred embodiment, the indentations 48 are located at approximately one centimeter intervals along the handle 46.

In this configuration, the proximal handle member 44 incorporates a detail member 50. The detail member 50 provides a means for the proximal handle member 44 to engage the indentations 48. As previously presented in FIG. 3, the detail member 50 similarly include a male mating configuration to facilitate a snap fit engagement process. The detail member 50 includes a male ridge member 52, which fits into a female depression 54 and can form a permanent bond therebetween.

The detail member 50 includes friction members 56, which facilitate engagement with at least one indentation 48 of a first series of indentations 48 along the proximal portion of the handle 46. A frictional drag force is created between the friction members 56 engaging at least one indentation 48 of a first series of indentations 48. It is contemplated that the proximal handle member 44 and the detail member 50 may be joined via alternative processes such as adhesive, solvent weld, thermal weld, ultrasonic weld, etc.

The friction members 56 may be, but are not limited to, protrusions such as semi-circular barbs. In a preferred embodiment, the friction members 56 engage at least one indentation 48 of a first series of indentations 48 and provide a clinician with a definitive depth measurement of the proximal handle member 44. Additionally, the friction members 56 serves to securely lock the proximal handle member 44 in place to provide a clinician with a consistent point of reference. It is contemplated that multiple friction members 56 may be employed. It is further contemplated that friction members 56 may have flexible portions, which may be of varying flexibility according to the particular requirements of the handle 46.

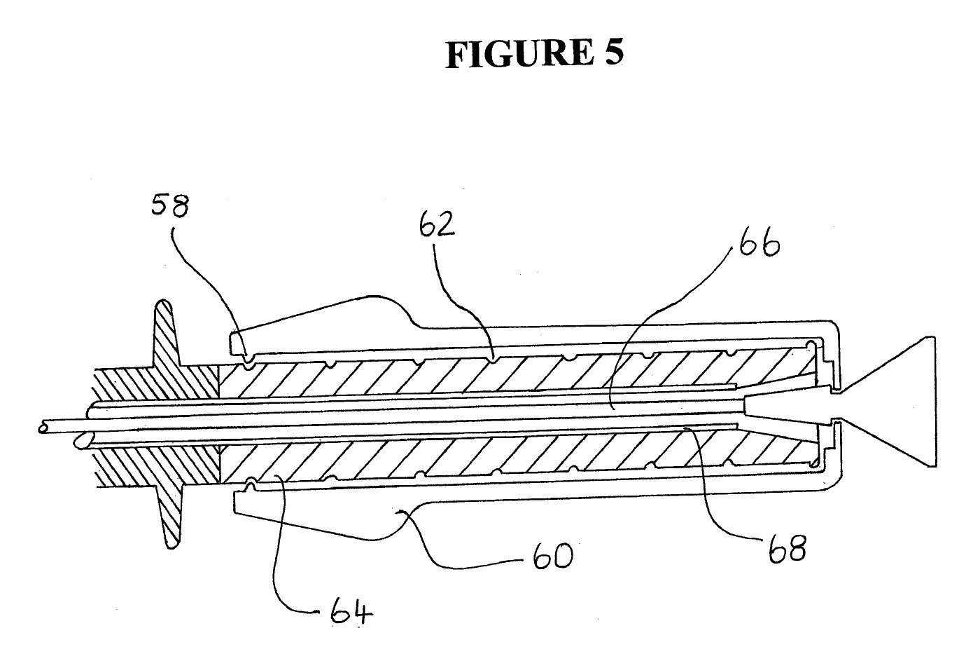

Referring to FIG. 5, a proximal portion of a fully assembled handle 64 is presented wherein a proximal handle member 60 can slideably advance a needle 66 within a sheath 68. In this configuration, friction members 58 are disposed to a distal portion of the proximal handle member 60 as semi-circular barbs. As presented, the friction member 58 allow the proximal handle member 60 to engage indentations 62 at any of a plurality of positions along the axis of the handle member 64. It is contemplated that each of indentation 62 can represent a specific length by which the needle 66 extends relative to the sheath 68. More specifically, in an engaged position, a clinician can set a maximum length by which the needle 66 can extend beyond the distal end of the sheath 68. A clinician may easily manipulate the position of the needle 66 by applying pressure to the distal portion of the proximal handle member 60. It is envisioned that an excessive level of pressure is not required to move the proximal handle member. However, such pressure must be sufficient to overcome the frictional resistance created between the friction member 58 and at least one indentation 62.

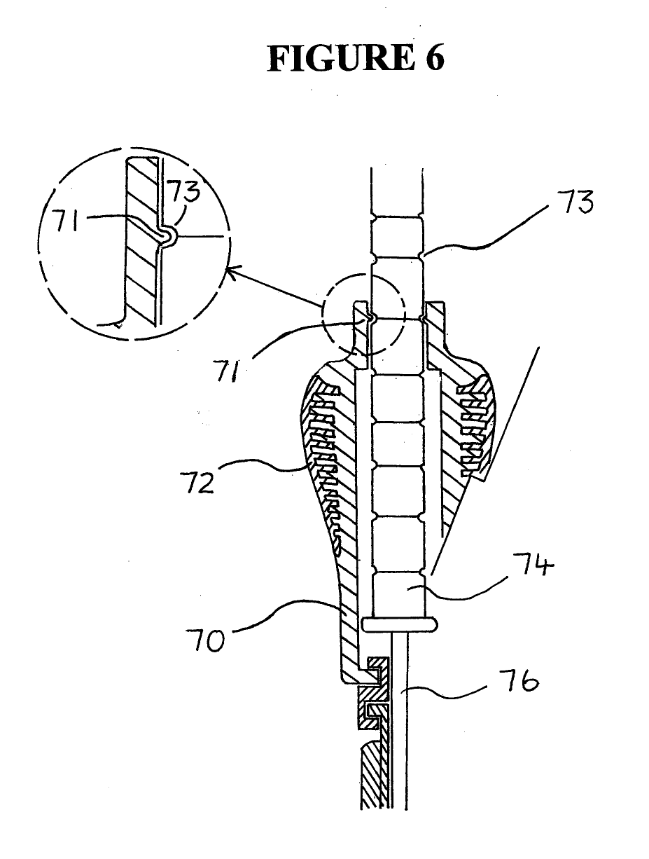

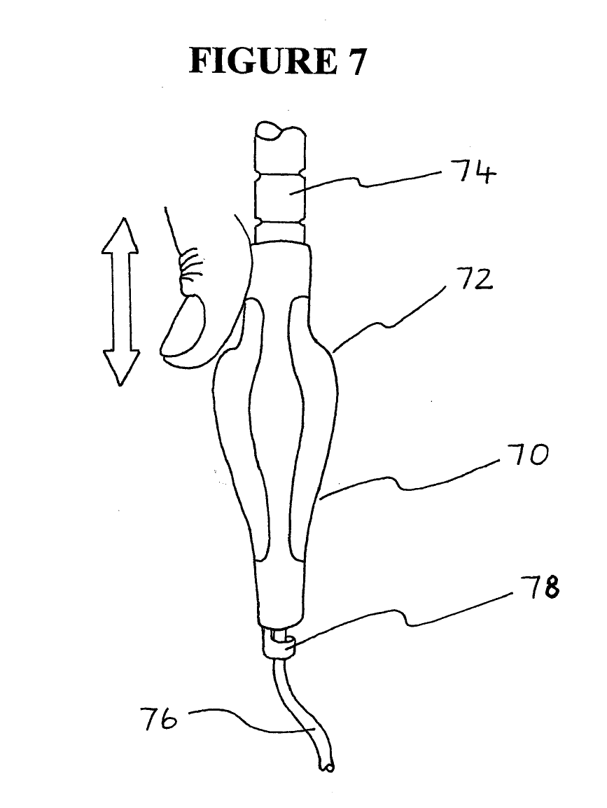

Referring to FIGS. 6-7, a distal handle member 70 is presented that is identical to the proximal handle member as described in FIG. 5. The distal handle member 70 includes friction members 71, which facilitate engagement with at least one indentation 73 of a second series of indentations 73 along the distal portion of a handle 74. A frictional drag force is created between the friction members 71, which engage at least one indentation 73 of a second series of indentations 73 along the handle 74.

The proximal handle member (not shown in Figure) and the distal handle member 70 further include a structural adaptation 72 that facilitates seamless movement along the handle 74. In the present configuration, the structural adaptation 72 has a larger outer diameter than other portions of the distal handle member 70. Additionally, the structural adaptation 72 is ergonomically configured to serve as a resting position for a finger or thumb of a clinician. It is contemplated that the structural adaptation 72 may provide a surface that facilitates movement of the distal handle member 70 along the handle 74. It is envisioned that the surface may be comprised of materials such as a rubber or other polymeric materials. The structural adaptation 72 may also provide a clinician with a tactile feel measurement system for gauging the position of the sheath 76 relative to the handle 74.

The distal handle member 70 also provides a means for engaging the needle biopsy device to another medical device. Referring to FIG. 7, the distal handle member 70 provides a connector 78 to facilitate attachment of the device to another medical device. The connector 78 is structurally capable of interacting with a connector on another medical device such as a channel or luer port. This interaction between the connector 78 and a connector on another medical device (not shown in Figure) can be, but is not limited to, a mating or locking connection.

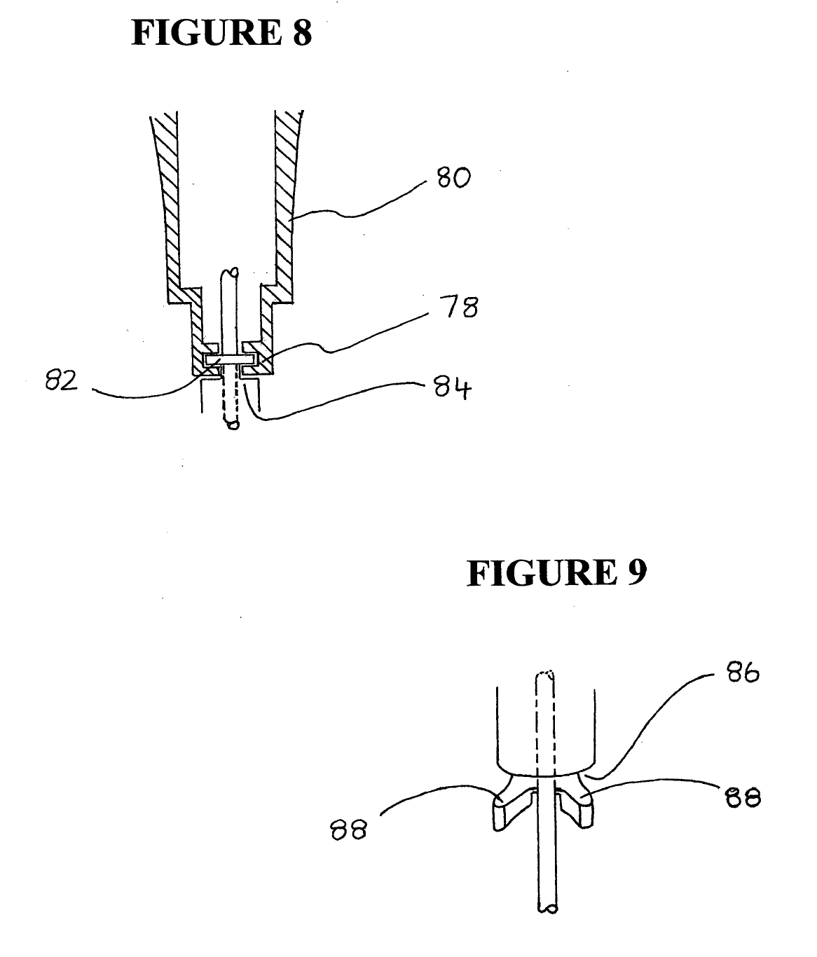

Referring to FIG. 8, an alternative embodiment of a connector 78 is shown. The connector 78 provides a mechanism for the quick connect and disconnect of a needle biopsy device 80 from a channel port 82 of a medical device 84. The connector 78 includes an adaptation that provides for connection relative to the longitudinal axis of the medical device. It is contemplated that the adaptation may be a female mating configuration and may further provide for a side loading removal motion of the device 80 from the channel port 82. It is further contemplated that the connector 78 is sized such that the device 80 is securely locked onto the channel port 82 in both an axial and perpendicular direction.

Referring to FIG. 9, another embodiment of a quick connect connector 86 is shown. The connector 86 includes two adaptations 88 that provide for connection relative to the longitudinal axis of the medical device. It is envisioned that the two adaptations 88 may represent a male mating configuration engaging a female mating channel port of another medical device. It is further envisioned that the two adaptations provide a secure connection to the medical device.

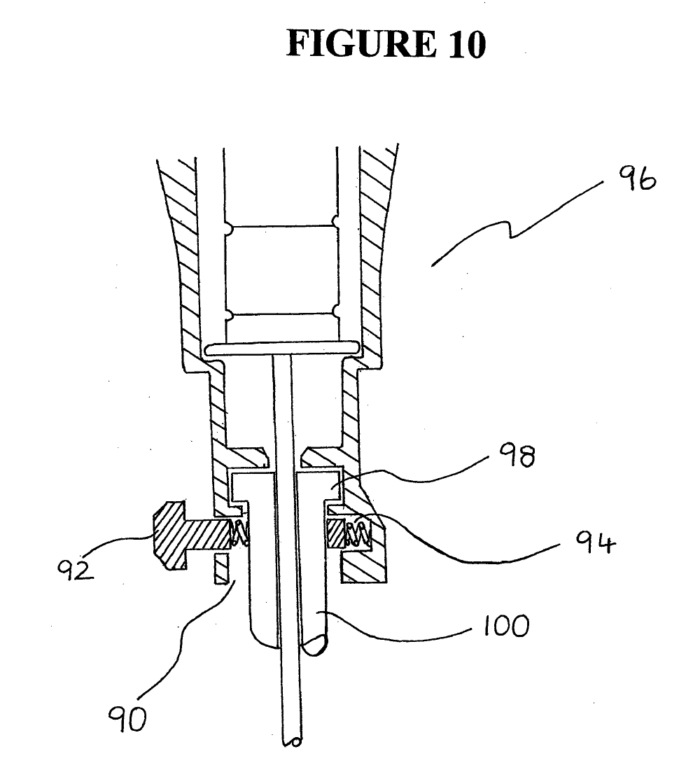

Referring to FIG. 10, another embodiment of a connector 90 is shown. The connector 90 is a spring loaded mechanism which facilitates connection to other medical devices with different channel ports. In the present configuration, a clinician can quickly load a device 96 axially onto a channel port 98 of another medical device 100. A button 92 is provided to work in concert with a spring 94 to provide a spring loaded tension between the device 96 and another medical device 100. The button 92 may also be depressed to release the spring loading tension and disengage the device 96. It is contemplated that the button 92 may be situated in a position to allow the clinician to utilize their thumb or finger to depress the button 92 without disturbing the desired configuration of the device 96.

Referring to FIG. 11, a distal portion of a handle 102 is presented wherein a connector 104 is joined via a snap fit process. It is contemplated that the connector 104 may utilize a snap fit detail 106, which can be a male mating configuration that engages a female mating configuration 108. In one embodiment, the snap fit detail 106 is permanently locked to the female mating member 108. It is further contemplated that the connector 104 may be adaptations in the form of two protruding male mating adaptations, a female mating adaptation, a spring loading mechanism, etc to satisfy the need for a quick connection mechanism.

Referring to FIGS. 1, 4, and 11, the needle biopsy device may also be assembled by engaging the connector 104 to the distal handle member 110, and subsequently attaching the distal handle member 110 to a stop portion 112. The stop portion 112 may be attached to the handle 46, as shown in FIG. 4, to complete the assembly of the handle 12, as shown in FIG. 1.

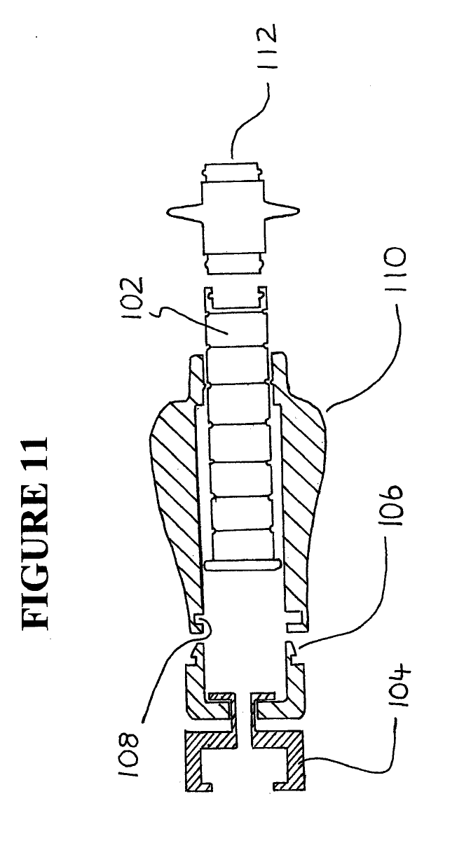

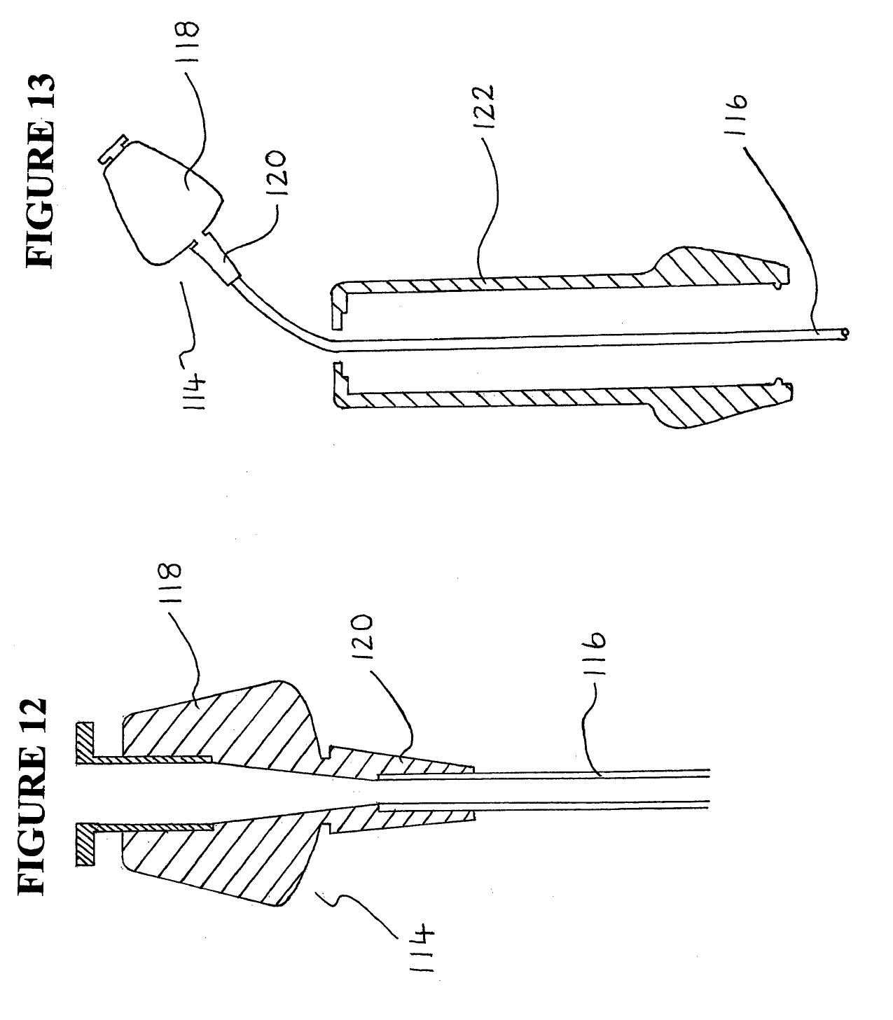

Turning to FIGS. 12 and 13, assembly of the needle biopsy device may be completed by inserting a needle housing member 114 into a proximal handle member 122. The needle housing member 114 is designed to allow a clinician to quickly and seamlessly remove the needle 116 after an aspirating sample is taken at a site of lesion or abnormality.

The needle housing member 114 includes a needle 116, a hub 118, and a strain relief 120. Due to the varying requirements of endoscopic ultrasound procedures, the needle 116 may be designed to range in length from fifty centimeters to two-hundred and fifty centimeters. Additionally, the needle 116 may be beveled via a single or double bevel at its distal end to aid a clinician in penetrating tissue in preparation of collecting an aspirated sample. It is contemplated that the needle 116 can be manufactured from several metallic based materials, such as stainless steel or alloys thereof and nitinol or alloys thereof. Alternatively, the needle 116 may be manufactured from polymeric materials including, but not limited to, polyetherkeytone, polyamide, polyethersulfone, polyurethane, ether block amide copolymers, polyacetal, polytetrafluoroethylene and derivatives thereof. Moreover, a combination of metallic based and polymeric materials may be suitable for this purpose. It is contemplated that one skilled in the art will realized that other materials suitable for manufacture in accordance with the present disclosure will also be appropriate.

The needle 116 requires a secure bond to the needle housing member 114. In one embodiment, the needle is attached to the needle housing member 114 via adhesive bonding. Although adhesive bonding is suitable for this purpose, an alternative and preferred method, such as direct injection over-molding can be utilized.

The method of over-molding consists of a two step molding operation with two constituent components. First, an inner component (not shown in the Figure) consists of a rigid polymer. The purpose of the inner component is to provide the primary bond between the hub 118 and the needle 116. It is contemplated that the inner component has shore hardness in the range of forty to eighty five Shore Durometer D. However, shore hardness in the range of seventy to eighty-five Shore Durometer D is generally preferable. It is contemplated that the shore hardness may include a scale of Shore Durometer A in addition to Shore Durometer D.

Second, the needle housing member 114 includes an outer component which consists of a strain relief 120. A common issue associated with prior art references is the kinking and deformation of needles during insertion and removal from a device. The strain relief 120 is designed to address the issue by providing a smooth transition and bend radius for the needle housing member 114 upon insertion and removal from the proximal handle member 112. The strain relief 120 is comprised of a relatively soft polymer, having shore hardness in the range of ten to fifty-five durometer. It is contemplated, however, that shore hardness in the range of thirty to forty-five durometer is preferable.

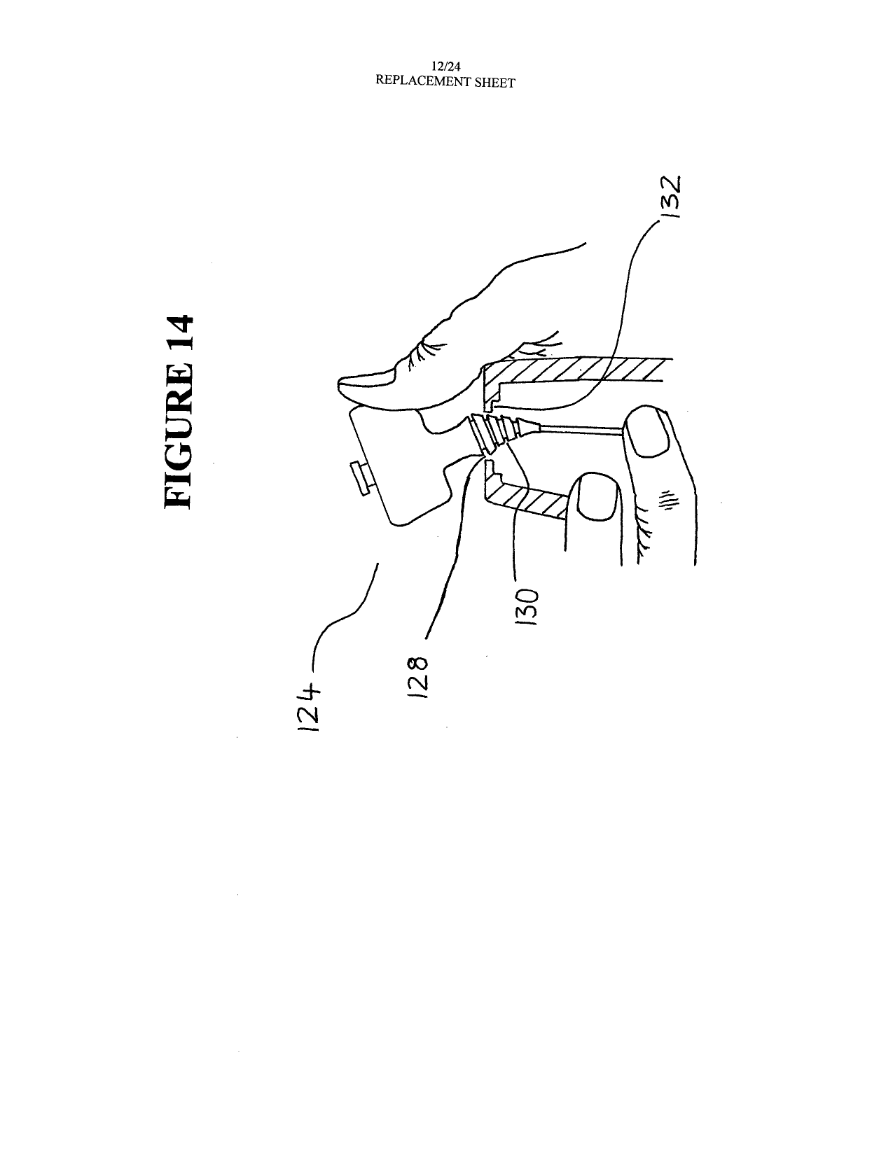

Referring to FIG. 14, an alternative embodiment of the needle housing member 124 is shown. In the present configuration, the needle housing member 124 is loaded into an opening at the proximal portion of a proximal handle member 126. To limit the need for a clinician to remove their hand from the device, the needle housing member 124 provides connecting details 128 that are immediately proximal to a strain relief 130 to facilitate insertion and removal of the needle housing member 124. More specifically, the connecting details 128 provides a means for rapid connection and disengagement of the needle housing member 124 relative to the proximal handle member 126. Upon inserting the needle housing member 124 into the proximal handle member 126, female connecting details 130 engage male connecting details 132 housed on the proximal handle member 126. The engagement of the female connecting details 130 and the male connecting details 132 provides the needle housing member 124 with a secure lock in the axial direction. This lock ensures that the needle housing member can not move or deform while is use.

The present configuration is designed to allow a clinician to easily disengage the needle housing member 124 from the proximal handle member 126. For example, once the clinician has acquired the desired tissue or fluid sample through needle aspiration, they may apply force in a substantially traverse direction to the needle housing member 124. The needle housing member 124 may be subsequently retracted for disposing the sample contained upon the needle. As a result, it is envisioned that a clinician can seamlessly acquire and insert another needle housing member 124 without reconfiguring the positions of the proximal handle member 126.

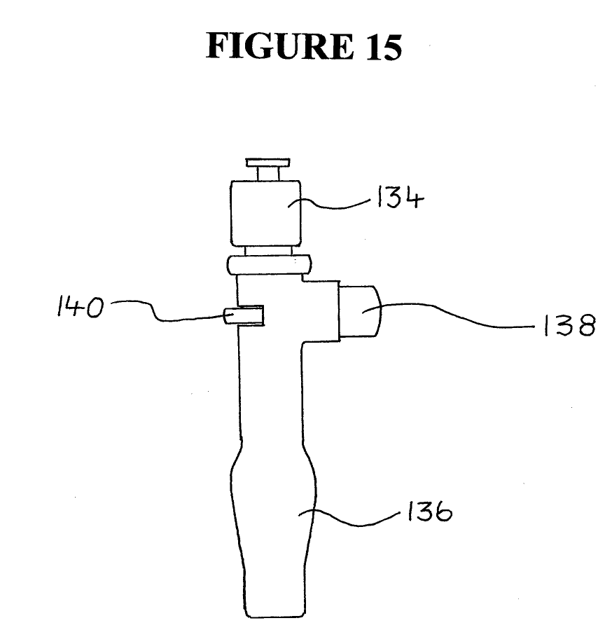

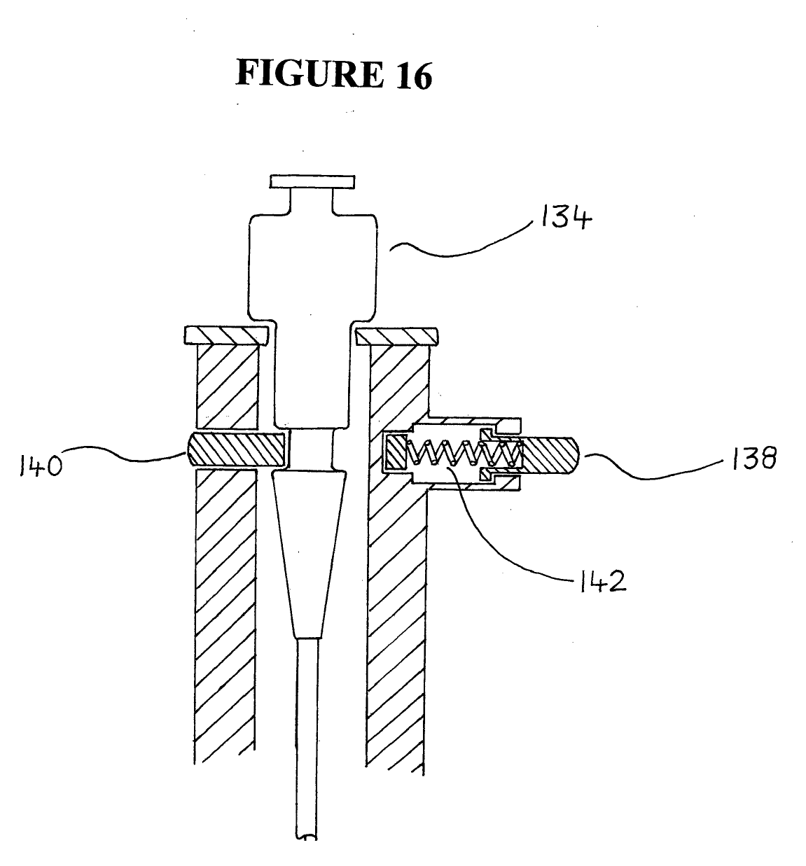

Referring to FIGS. 15 and 16, it is contemplated that a spring loaded mechanism may be provided to facilitate the removal of a needle housing member 134 from a device 136. In the present configuration, a release member 138 is provided which functions in concert with a lever 140. The lever 140 operates under a spring loaded tension 142 to securely fasten the needle housing member 134 to the device 136. The lever 140 is operated by depressing the release member 138. Upon depressing the release member 138, the tension released by a spring 142 causes the lever 140 to release the needle housing member 134 from the device 136.

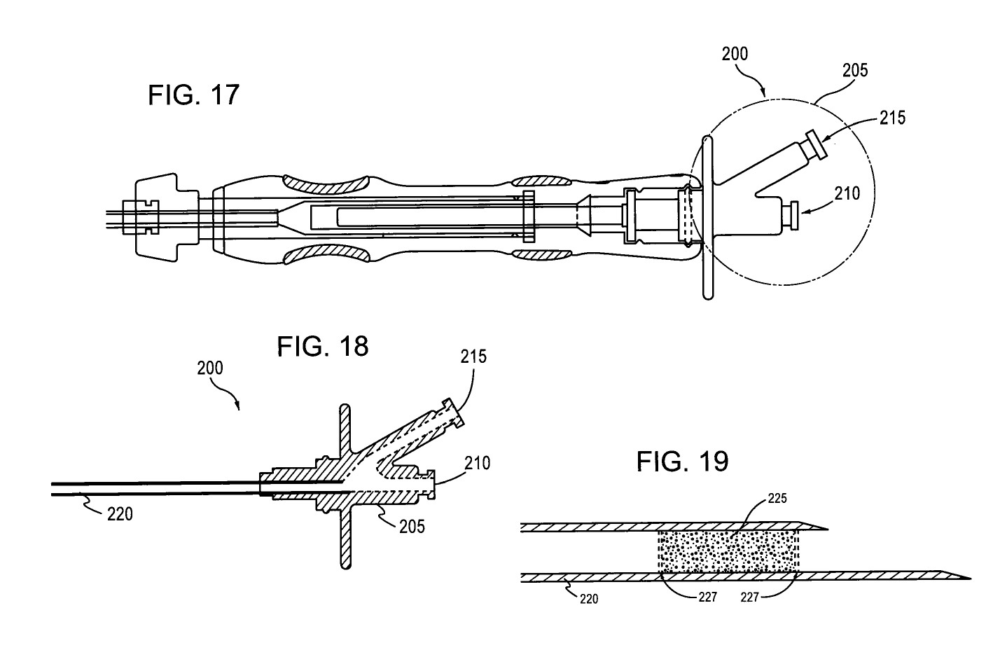

A further embodiment of the needle housing member of the biopsy devices of the invention is illustrated herein in FIGS. 17 and 18 respectively. In this instance, the proximal hub 205 design of the needle housing member 200 consists of a bifurcation or Y-Body design to provide for a primary port 210 (sometimes referred to herein as a luer port) through which a stylet 212 or similar device is inserted, and a side port 215. This bifurcated hub may be over-molded onto the proximal end of the needle component 220 or alternately may be thermally bonded in position or alternately may be attached to the proximal needle end via adhesive bonding techniques. Most preferably the needle hub is manufactured from a rigid or semi-rigid polymer as before disclosed. The provision of an additional side port 215 on the bifurcation provides means for the administration or injection of a fluid media to the targeted tissue site under diagnosis.

The needle housing member may alternately be pre-loaded at the distal end, with the desired agent to be administered. For example, without limitation, the distal end of the needle component 220 of the needle housing member may be pre-loaded with an agent 225 (e.g., a therapeutic agent or imaging agent) encapsulated in a membrane 227 or capsule 229, or in the form of a pellet or a seed 231; one or more fiducial markers; and/or one or more biomarkers. The needle component of the needle housing member may also be pre-loaded at the distal end with a desired device, such as an imaging device (e.g., a probe), a marker (e.g., a fiducial marker or a biomarker), a pacing lead, or a stent. In this instance, the physician will perform the diagnostic procedure as previously described. Having aspirated the sample from the desired anatomical location/tumor mass, the physician would then remove the needle housing member from the proximal handle housing, and replace it with needle housing member having the desired agent, reagent, marker, and/or device pre-loaded at the distal end.

Exemplary embodiments of devices of the invention configured for delivering an agent, reagent, marker, and/or device in conjunction with an EUS or EBUS procedure are described in further detail below.

As shown in FIG. 17 of U.S. patent application Ser. No. 12/243,367 (published as US2010/0081965, incorporated by reference herein, a sheath lumen 144 is provided to house the needle 22 from the proximal handle member 14 through the distal handle member 16, as shown in FIG. 1 herein. The sheath lumen 144 is comprised of, but not limited to, thermoplastic materials. It is contemplated that the thermoplastic materials may be polyurethane, polyamide and derivatives thereof, ether block amide copolymers, polyimide, placental, polyethylene and derivates thereof, polytetrafluoroethyelene, and the like. In a preferred embodiment, the sheath lumen 144 is comprised of a heliacally braided configuration 146 of outer thermoplastic materials with a lubricious inner core 148.

The inner core 148 may be made from polytetrafluoroethyelene, fluorinated ethylene propylene, or derivatives thereof, to provide a lubricous surface for the needle 22, as shown in FIG. 1 herein, as it is passed through the sheath lumen 144. It is contemplated that the sheath lumen 144 may have an outer diameter ranging from three French to twelve French. It is further contemplated that the sheath lumen 144 may have an inner diameter ranging from two French to ten French. In a preferred embodiment, the inner and outer diameter of the sheath 144 is between three French and six French.

As shown in FIG. 18 of U.S. patent application Ser. No. 12/243,367 (published as US2010/0081965), incorporated by reference herein, a taper 152 on the distal end of a needle 150 may be provided to provide a level of interference between a sheath 154 and the needle 150 during needle advancement. The taper 152 addresses the issue of needle instability by providing an enlarged portion that provides a frictional resistance in the form of a drag force. It is envisioned that the taper 152 may be incorporated onto the needle 150 through centerless grinding or cold-drawing techniques.

As shown in FIG. 19 of U.S. patent application Ser. No. 12/243,367 (published as US2010/0081965), incorporated by reference herein, an alternative embodiment is presented wherein a needle 156 comprises stabilizing bulbs 158 located at constant increments over the length of the needle 156. These bulbs 158 may be spaced anywhere from two millimeters to one centimeter apart and may be located over the entire length of the needle 156 or over a portion of the needle 156. It is contemplated that the bulbs 158 may be circular or elliptical in geometry and may be incorporated onto the needle 156 via soldering or laser welding or incorporating into the grind profile of the needle 156. It is further contemplated that the stabilizing bulbs 158 will provide sufficient frictional resistance between the needle 156 and a sheath 160.

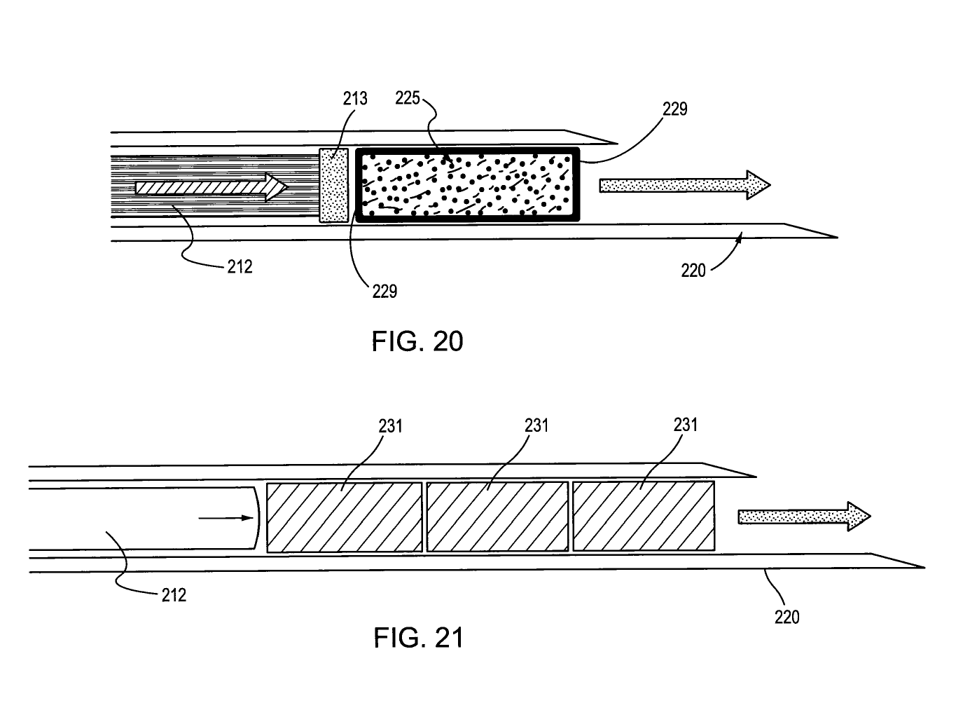

As shown in FIG. 20 of U.S. patent application Ser. No. 12/243,367 (published as US2010/0081965), incorporated by reference herein, another embodiment is contemplated wherein a series of barbs 162 are located at varying intervals along the length of a needle 164. The purpose of the barbs 162 is to reduce the effective clearance between the outer diameter of the needle 164 and the inner diameter of a sheath 166. It is contemplated that the barbs 162 may be positioned at the distal end of the needle 164 or alternately, may be spaced over the entire length of the needle 164.

It is contemplated that all forms of protrusions, including the “taper”, “bulb” or “barb” details, extend into the sheath 166 when the needle 164 is fully extended relative to the sheath 166. This ensures that at maximum needle insertion depth, the needle 164 is kept stable in the assembly and achieves the desired design intent.

As shown in FIG. 21 of U.S. patent application Ser. No. 12/243,367 (published as US2010/0081965), incorporated by reference herein, a clinician may yield the benefit of improving the echogenicity and ultrasonic visibility of a needle 168 during endoscopic ultrasound, by enhancing the definition of the needle 168 and the ability to discern needle 168 during the procedure. It is contemplated that the needle 168 can be surrounded by echogenic materials such as a polymer impregnated with sonically reflective particles to provide ultrasonic visibility. It is further contemplated that ultrasonic visibility may be, but is not limited to, x-rays, ultrasounds, sonography, etc. It is envisioned that the polymer may be, but is not limited to, a thermoplastic or thermoset coating. It is further contemplated that the echogenic properties of the needle 168 may be enhanced through techniques such as sandblasting, laser etching, surface roughening, the introduction of various patterned geometries onto the surface of the needle, etc.

In the present configuration, an alternative configured is contemplated wherein a polymeric sleeve or jacket 170 covers the proximal portion of the needle 160, which extends distally from a sheath 172 back to a hub on a housing member 174. The purpose of the sheath 172 is to act as a “buffer-layer” between the outer diameter of the needle 168 and the inner diameter of the sheath 172. In this way, the advancement of smaller diameter needles are stabilized as a result of frictional resistance between the needle 168 and the sheath 172. The material used for the needle jacket 170 is preferably extruded from a thermoplastic material such as polyurethane, polyethylene, polypropylene or copolymers thereof, polyamide, polyimide, and polyether block amide or copolymers thereof. Alternately and more preferably, the jacket 170 may be extruded from a highly lubricious material such as polytetrafluoroethylene or fluorinated ethylene-propylene. It is contemplated that by utilizing low co-efficient of friction materials on the outer wall of the needle 168, the frictional drag or insertion force required to insert the needle 168 through the sheath 172 to the desired anatomical location for aspiration is minimized.

In the present configuration, the polymeric jacket or sleeve 170 is located to commence at the needle housing member 174 and run the entire length of the needle 168 to a specified location. This method ensures that the distal portion of the needle 168, which extends from the sheath 172, is bare and the polymeric jacket 170 does not interfere with passage of the needle 168 through the clinical anatomical mass under evaluation. The jacket 170 may be captured at the proximal end during insert molding of the needle housing member 174 or alternately may abut the needle housing member 174.

The incorporation of such a polymeric jacket 170 to encase the proximal portion of the needle 168 also serves to provide the clinician with passive feedback during removal of the needle 168 from the proximal handle housing. During removal of the needle 168 from the device once the sample has been acquired, it is important that the clinician be made aware of when they are approaching the sharp end of the needle 168. With the polymeric jacket 170 being positioned at a constant distance from the sharp bevel of the needle 168, once the clinician observes the end of the polymeric jacket 170 on the needle 168, they are passively made aware that a sharp bevel 176 is located at a specified distance from the end of the polymeric jacket 170. This passive feedback is important as the clinician can now exercise additional caution to ensure that they do not inadvertently pierce themselves with the needle 168 or cause the needle 168 to become entangled, endangering the diagnosing value of the collected sample.

It is contemplated that these concepts pertain to the maintenance of stability during needle advancement, particularly in the case of a needle 168 with 22 or 25 AWG, wherein the gap between outer diameter of the needle 168 and inner diameter of the sheath 172 is more appreciable. It is desirable to also incorporate the jacket type arrangement into the design for the 19 AWG needle portion. With a reduced amount of concentric clearance available between inner diameter of the sheath 172 and the outer diameter of the needle 168 in the case of a 19 AWG needle 168, the polymer jacket 170 may take the form of polytetrafluoroethylene or other thermoplastic material heat shrink which is thermally laminated onto the outer diameter of the needle 168. Alternately, it is further contemplated that a 19 AWG needle 168 may be spray coated with a lubricious material such as teflon. At the distal end of the needle 168, the heat shrink material or coated material may terminate at specific distance from the sharp end of the needle 168. It is envisioned that this method will provide the clinician with feedback as to when they are approaching the sharp bevel at the distal end during extraction of the needle 168.

### Delivery of Therapeutic Agents

In the embodiment where the devices of the invention have a bifurcation in the hub of the needle housing member, such devices can facilitate efficient delivery of one or more desired therapeutic agents to a targeted sites in the gastrointestinal and/or respiratory tracts. For example, without limitation, devices of the invention having a bifurcated hub in the needle housing member can be used to deliver chemotherapeutic agents, tumor necrosing factors and growth factors to desired sites in the gastrointestinal or respiratory tracts, to provide a localized therapeutic effect in the treatment of benign and malignant tumors.

As previously described, the needle is advanced to the intended anatomical location and the stylet component is removed. A syringe or other injection system may be attached to the side port of the bifurcation in the hub of the needle housing member, and various agents or reagents such as, but not limited to, chemotherapeutic agents/gels, sclerosing and necrosing agents, growth factors, radiation liquids, and/or imaging agents may be injected there-through. Alternately, such a bifurcated embodiment may be beneficial in the delivery of fibrin glues to targeted sites (e.g., tumor sites). Such fibrin glues or gels may be radioactive in nature (for example, they may contain a β-radiation emitting rhenium-188/rhenium-186 suspended in the gel which provides for an effective method of delivering high doses of local radiation to tumor tissue, particularly to wet areas where high adhesive strength and long-term radiation (with or without drug) delivery are needed.

Alternatively, the desired agent can be pre-loaded into the distal end of the needle component of the needle housing member for direct delivery to the targeted tissue site in the gastrointestinal and/or respiratory tract. In an exemplary embodiment where a user wishes to administer an agent, such as a therapeutic agent, directly to the diseased site, a needle housing member with pre-loaded therapeutic agent 225 encapsulated in a membrane 227 at the distal end of the needle component (see e.g., FIGS. 19 and 20) is loaded into the catheter system of the FNA device and advanced to exit the distal end of the catheter sheath. The needle 220 is advanced into the targeted tissue site, such as a tumor or mass. A syringe may be attached to the side port 215 in the proximal needle hub assembly and pressure applied to the system. Under the application of this pressure, the encapsulating membrane 227 breaks and the enclosed agent 225 is dispensed into the targeted tissue site.

It is desirable that the encapsulating membrane be manufactured from an inert material such as a thermoplastic polymer or rubber which is easily deformed under the application of an applied load from the proximal end, but which has highly chemical resistant properties to avoid breakdown by the pre-loaded, encapsulated agent housed therein.

An alternate embodiment to facilitate delivery of one or more agents 225 to sites in vivo is illustrated in FIG. 20. In this instance, the agent is encapsulated in a capsule 229. The capsule in this instance may be deposited at the desired site by re-inserting the stylet 212 into the needle housing member from the proximal end. The stylet may optionally include a plunger 213 at distal portion thereof. The stylet is advanced to push the capsule 229 out the distal end of the needle 220 at the desired location. It is preferable that the outer “shell” of the capsule 229 be capable of degrading or being absorbed by the body analogous to the mode of operation of orally administered tablets common is the field. Examples of such materials of polymers such as Poly Vinyl Alcohol (PVOH), polyglycolide/poly(glycolic acid)/poly(lactic acid) (PGLA/PLA), homopolymers, oligomers and copolymers thereof, polyanhydrides, and polyethylene glycol and blends thereof.

Chemotherapy, in its most general sense, refers to treatment of disease by chemicals that kill cells, specifically those of micro-organisms or cancer. In popular usage, it will usually refer to antineoplastic drugs used to treat cancer or the combination of these drugs into a cytotoxic standardized treatment regimen as opposed to targeted therapy.

Targeted therapy is a type of medication which blocks the growth of cancer cells by interfering with specific targeted molecules needed for carcinogenesis and tumor growth, rather than by simply interfering with rapidly dividing cells. Targeted cancer therapies may be more effective than current treatments and less harmful to normal cells. The main categories of targeted therapy are small molecules and monoclonal antibodies. Some examples of “small molecule” targeted therapies used in the treatment of gastrointestinal and lung cancer tumors include, for example, Imatinib Mesylate, Gefitinib (which targets various epidermal growth factor receptors [EGFR's]—FDA approved for small cell lung cancer treatment), Erlotinib, and Bortezomib. Many cancers may also be treated via surgery in conjunction with cytotoxic chemotherapeutic drugs such as vincristine, cisplatin, vinblastine, methotrexate, and 5-fluorouracil (5-FU), as examples. Some examples of monoclonal antibodies used in the field of cancer treatment include Cetuximab (which targets the epidermal growth factor receptor; used in the treatment of colon cancer and non-small cell lung cancer) and Bevacizumab (this drug is approved for use in the treatment of colon cancer, breast cancer and non-small cell lung cancer). Other examples include trastuzumab (Herceptin), and rituximab.

Alternatively, a number of cancer treatment drug compositions are available which interfere with microtubule function, interrupting cell division and multiplication, Examples of such agents are described in U.S. Pat. No. 6,544,544 B2, the contents of which are hereby incorporated by reference in its entirety, and further include those referenced above, in addition to paclitaxel, estramustine, colchicine, methotrexate, curacin-A, epothilone, vinblastine, or tBECV.

Antimetabolites can be used in cancer treatment, as they interfere with DNA production and therefore cell division and the growth of tumors. Because cancer cells spend more time dividing than other cells, inhibiting cell division harms tumor cells more than other cells.

Anti-metabolites masquerade as purine (azathioprine, mercaptopurine) or pyrimidine which become the building blocks of DNA. They prevent these substances becoming incorporated in to DNA during the S phase (of the cell cycle), stopping normal development and division. An example of an antimetabolitic agent is fluorouracil. It resembles a normal cell nutrient needed by cancer cells to grow. The cancer cells take up fluorouracil, which then interferes with their growth by interfering with conventional cancerous cell division mechanics.

Further research in the field has seen the use of nanoparticles emerge as a useful vehicle for the delivery of poorly-soluble chemotherapy agents such as paclitaxel in the treatment of cancer. Protein-bound paclitaxel (e.g., Abraxane) or nab-paclitaxel was approved by the US FDA in January 2005 for the treatment of refractory breast cancer, and allows reduced use of the Cremophor vehicle usually found in paclitaxel. Nanoparticles made of magnetic material can also be used to concentrate agents at tumor sites using an externally applied magnetic field. Specially-targeted delivery vehicles aim to increase effective levels of chemotherapy for tumor cells while reducing effective levels for other cells. This should result in an increased tumor kill and/or reduced toxicity.

Necrosis is the name given to unnatural death of cells and living tissue. It begins with cell swelling, chromatin digestion, disruption of the plasma membrane and organelle membranes. Late necrosis is characterized by extensive DNA hydrolysis, vacuolation of the endoplasmic reticulum, organelle breakdown, and cell lysis. The release of intracellular content after plasma membrane rupture is the cause of inflammation in necrosis.

Tumor Necrosis factors (TNF) acts via the cellular TNF Receptor (TNF-R) and is part of the extrinsic pathway for triggering apoptosis. TNF-R is associated with procaspases through adapter proteins (FADD, TRADD, etc.) that can cleave other inactive procaspases and trigger the caspase cascade, irreversibly committing the cell to apoptosis. TNF interacts with tumor cells to trigger cytolysis or cell death. TNF can interact with receptors on endothelial cells, which leads to increased vascular permeability allowing leukocytes access to the site of infection. This is a type of localized inflammatory response.

There are various types of TNF factors which may be administered to cancerous tumor sites to disrupt cellular metabolism mechanics. Tumor necrosis factor-alpha (TNF-α) is the most well-known member of this class, and sometimes referred to when the term “tumor necrosis factor” is used. Tumor necrosis factor-beta (TNF-β) is a cytokine that is induced by interleukin 10. Various sclerosing agents (e.g. alcohol) may also be delivered to the tumor site to effectively “kill” the tumor, preventing further cell division, essentially “drying out” cells within same. The most popular sclerosants used today are alcohol, bleomycin, OK-432 (which is also known as Picibanil), Ethibloc, as well as 3% sodium tetradecyl sulfate.

It may also be desirable to deliver various growth factors (i.e. a factor responsible for regulating cell proliferation, development, migration, differentiation and/or activity) to areas of the gastrointestinal or respiratory systems to promote cellular growth or regeneration. The term “Growth Factors” generally refers to a naturally occurring human protein capable of stimulating cellular growth, proliferation and cellular differentiation. Growth factors are important for regulating a variety of cellular processes. Growth factors typically act as signaling molecules between cells. Examples are cytokines and hormones that bind to specific receptors on the surface of their target cells.

They often promote cell differentiation and maturation, which varies between growth factors. For example, bone morphogenic proteins stimulate bone cell differentiation, while fibroblast growth factors and vascular endothelial growth factors stimulate blood vessel differentiation (angiogenesis). These growth factors are typically targeted to tumor-associated endothelial cells, and generally act by binding to a growth factor receptor on the surface of the targeted tumor-associated endothelial cell.

Individual growth factor proteins tend to occur as members of larger families of structurally and evolutionarily related proteins. There are dozens and dozens of growth factor families such as TGF-beta (transforming growth factor-beta), VEGF/VPF (vascular endothelial growth factor/vascular permeability factor), FGF (fibroblast growth factor), pleitotrophin, BMP (bone morphogenic protein), neurotrophins (e.g., NGF, BDNF, and NT3), fibroblast growth factor (FGF), and so on.

Several well known growth factors, some of which are mentioned above, include but are not limited to: transforming growth factor beta (TGF-β), granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), and hepatocyte growth factor (HGF).

### Delivery of Radiation Therapy

Devices of the invention having a bifurcation in the hub of the needle housing member may also be used to deliver radiation therapy in a liquid, gel, or glue form directly to a targeted tissue site (e.g., tumor or mass) in the respiratory and/or gastrointestinal tracts to treat various cancerous or other tumors in the manner previously described. Alternatively, the distal end of the needle component 220 of the needle housing member can be pre-loaded with radiation therapy in the form of a radioactive seed or pellet 231, as shown in FIG. 21. The radioactive seed or pellet can be directly delivered to targeted locations in the gastrointestinal and/or respiratory tracts using a syringe or stylet 212 to push the pellet/seed 231 out the distal end of the needle 220 at the desired location, in the manner previously described.

Brachytherapy (internal radiation) is a method used in the medical arena to administer radiation therapy for treating various cancers. The modality uses radioactive materials delivered close to tumors to kill cancer cells while minimizing damage to surrounding normal healthy tissues. There are two main subtypes of brachytherapy. Interstitial radiation therapy places radioactive material into tissue, within or near the cancer. Intracavitary radiation therapy places the radioactive material into a body cavity (E.g. The chest cavity, abdominal cavity or vagina) near a cancerous growth. In contrast, external beam radiation therapy or teletherapy uses radiation delivered to a cancer from outside of the body. Brachytherapy allows a physician to use a higher total dose of radiation to treat a smaller area and in a shorter time than is possible with external radiation treatment.

Permanent brachytherapy, also called seed implantation, involves placing radioactive seeds or pellets in or near the tumor and leaving them there permanently. After several weeks or months, the radioactivity level of the implants eventually diminishes to negligible levels. The seeds then remain in the body, with no long term adverse effect on the patient.

The first treatments of this kind used needles containing Radium-226 in the treatment of prostate cancer but modern methods tend to use Iridium-192 as the radioactive source.

### Delivery of Fiduciary Markers

The devices of the invention can be used to deliver one or more fiduciary markers (sometimes referred to herein as fiducial markers) to desired sites in the gastrointestinal or respiratory tracts, to provide “landmarks” in the anatomy to aid in directed radiotherapy or other therapy.

As with the direct delivery of an encapsulated therapeutic agent or radioactive seed or pellet (e.g., shown in FIGS. 19-21), a similar mode may be adopted to deposit fiducial markers at various sites within the anatomy. The distal end of the needle component 0 of the needle housing member is pre-loaded with one or more fiducial markers. It is preferable that these fiducial markers be made out of gold and/or carbon materials so that they are readily visible and biocompatible as a long or short term implant and are readily visible under fluoroscopy and/or other imaging techniques, as previously described. FIG. 21 illustrates a needle housing member in which the distal end of the needle component 220 thereof has been preloaded with three fiducial marker components 231. Alternately the needle component of the needle housing member may be pre-loaded with a single fiducial marking component 231. A stylet 212 is advanced to push the fiducial marker or markers 231 to deposit out of the distal end of the needle 220 at the desired location.

The fiducial marker component may vary in diameter (e.g., from 0.2 mm to 4 mm), but is preferably in the range of 0.5 mm-1.2 mm in diameter. The fiducial marker component may vary in length (e.g., from 0.5 mm to 6.0 mm, preferably in the range of 1.0 mm-3.5 mm in length). The fiducial components may alternately be circular, square or rectangular in cross section.

Fiduciary markers are used in a wide range of medical imaging applications. Images of the same subject produced with two different imaging systems may be correlated by placing a fiduciary marker in the area imaged by both systems. In this case, a marker which is visible in the images produced by both imaging modalities must be used. By this method, functional information from Single photon emission computed tomography (SPECT) or Positron emission tomography (PET) can be related to anatomical information provided by Magnetic Resonance Imaging (MRI). Similarly, fiducial points established during MRI can be correlated with brain images generated by magnetoencephalography to localize the source of brain activity.

In the field of electrophysiology, fiducial points are landmarks on the ECG complex such as the isoelectric line (PQ junction), and onset of individual waves such as PQRST.

In radiotherapy and radiosurgical systems such as the CyberKnife™ technology (Accuray Inc. Sunnyvale, Calif.) fiducial points are landmarks introduced into a tumor (soft tissue or other) to facilitate correct targets for treatment. Small markers (fiducials) made out of gold and/or carbon for improved biocompatibility properties and high density to give good contrast on X-ray images are surgically implanted in the patient. This is usually carried out by an interventional radiologist, or neurosurgeon. The placement of the fiducials is a critical step if the fiducial tracking is to be used. If the fiducials are too far from the location of the tumor, or are not sufficiently spread out from each other it will not be possible to accurately deliver the radiation. Once these markers have been placed, they are located on a CT scan and the image guidance system is programmed with their position. When X-ray camera images are taken, the location of the tumor relative to the fiducials is determined, and the radiation can be delivered to any part of the body in a more localized and efficient manner. Fiducials are known however to migrate and this can limit the accuracy of the treatment if sufficient time is not allowed between implantation and treatment for the fiducials to stabilize.

### Delivery of Biomarkers

The devices of the invention can be used to deliver one or more biomarkers that may be used as part of an image guided system in conjunction with a fine needle aspiration (FNA) using ultrasound guided imaging techniques. Such biomarkers are used provide an identifying position for the delivery of subsequent therapy during patient treatment.

A similar mode may be adopted to deposit biomarkers at various sites within the anatomy, enabled through the use of FNA techniques and more specifically, in conjunction with the present catheter and needle invention disclosed herewith. For example, to implant the biomarker at the intended site, in conjunction with the EUS and/or EBUS procedure, the stylet is advanced to push the biomarker or markers to deposit out of the distal end of the needle at the desired location, such as in the manner shown in FIG. 21, which illustrates a needle housing member in which the distal end of the needle component 220 thereof has been preloaded with three fiducial markers (or alternately, biomarker) components 231. Alternately the needle component of the needle housing member may be pre-loaded with a single biomarker marking component 231. Once implanted, the biomarker may be used in conjunction with EUS and/or EBUS and/or other imaging techniques, or x-ray to improve location and detection capabilities for further therapeutic administration.

The biomarker component may vary in diameter (e.g., from 0.2 mm to 4 mm, preferably in the range of 0.5 mm-1.2 mm in diameter). The biomarker component may also vary in length (e.g., from 0.5 mm to 6.0 mm, preferably in the range of 1.0 mm-3.5 mm in length). Suitable biomarkers for use on conjunction with the devices of the invention may include a MEMS (MicroElectroMechanicalSystem) housing manufactured from a biocompatible material. Once delivered and implanted at the desired site, the biomarker incorporating the MEMS may be tracked via endoscopic or endobronchial ultrasound to provide a more precise location for follow-up directed therapy. The MEMS housing may, for example, may take the form of a silicone chip which incorporates a biocompatible outer surface. It is possible to manufacture the MEMS housing component using MEMS manufacturing techniques that are known to those skilled in the art. In alternate embodiments of the biomarker component, it is preferable that the biomarker material be manufactured from an ultrasound resonant material such as glass, or polymer. Alternately, the biomarker component may be manufactured from a material which is biocompatible and readily visible under fluoroscopy such as metals (for example, stainless steel, nickel, titanium, tantalum and alloys thereof) metal filled and/or metallic filled (for example tungsten, barium, bismuth subcarbonate, bismuth-oxychloride, etc.) polymer materials.

Other suitable biomarkers for use in the devices of the invention are described in U.S. Pat. Nos. 6,654,629, 6,161,034, 5,281,408, and 5,636,255, the contents of which are each incorporated by reference herein in their entireties. As outlined by Montegrande et al., U.S. Pat. No. 6,654,629, the first class of prior art biomarkers include materials that have different ultrasound reflective properties and only remain in the body temporarily, eventually being reabsorbed by the body. An example of this technology is shown in Burbank et al., U.S. Pat. No. 6,161,034, assigned to SENOREX®, that teaches detectable markers that may be introduced by a cavity created by removal of a biopsy specimen to mark the location of the biopsy site so that it may be located in a subsequent medical/surgical procedure. The marker preferably includes gasses, saline solutions, or similar materials. The markers remain present in sufficient quantity to permit detection and location of the biopsy site at the first time point (e.g., 2 weeks) after introduction but clear from the biopsy site or otherwise not interfere with imaging of tissues adjacent the biopsy site at a second time point several months after introduction.

Unger et al, U.S. Pat. No. 5,281,408, identifies substantially homogeneous aqueous suspensions of low density micro-spheres which are presented as contrast media for imaging the gastrointestinal tract and other body cavities using computed tomography. In one embodiment, the low density microspheres are gas-filled. With computed tomography, the contrast media serve to change the relative density of certain areas within the gastrointestinal tract and other body cavities, and improve the overall diagnostic efficacy of this imaging method.

Ellis, U.S. Pat. No. 5,636,255, describes a method and system for correlating accuracy of computer tomography (CT) image resolution. Small radio-opaque markers having a diameter less than one slice width of a CT scan are embedded in the object, such as a bony skeletal member, to be measured, the object is then CT scanned so that the radio-opaque markers appear in at two slices of the scan. The markers are also physically located by detecting them with a sensor, such as a positioning pointer. Also described is one form of marker, comprising a tantalum sphere mounted in a ceramic, preferably alumina, pin.

Foerster et al, U.S. Pat. No. 5,902,310, illustrates an implantable marking device which is designed to percutaneous delivery of permanent markers to desired tissue locations within a patient's body, even if the desired locations are laterally disposed relative to the distal end of the delivery device, as is the case for conduit or cavity walls. This provides several advantages to the physician in diagnosis and management of tissue abnormalities, such as a means of localization of a tissue abnormality for follow-up surgical treatment, and a means of tissue abnormality site identification for purposes of ongoing diagnostic follow-up. In one preferred construction, a radiographic clip is configured in the form of a surgical staple. A disposable tissue marker applier, which comprises a flexible tube, pull wire, and squeeze handle, is employed to advance and deploy the clip to a desired tissue location. Either a flexible or a rigid introducer is also provided for providing access to the site to be marked.

Once the biomarker is planted in the desired site, it can be imaged using an appropriate imaging system. In the case of fluorescence microscopy, certain molecules, by virtue of their chemical structure, have the ability to emit light of a specific wavelength following absorption of light of a shorter, higher energy wavelength. This process of light absorption and re-emission is termed ‘fluorescence’ and the molecules which exhibit this behavior are termed ‘fluorochromes’. All fluorochromes have characteristic light absorption and emission spectra. Upon absorption of photons of the excitation wavelength, fluorochromes become excited into a higher, unstable energy state. This instability is then relieved by the subsequent production of photons of a lower energy emission wavelength. For example, fluorescein, a commonly used fluorescent dye—absorbs blue light and emits green light. The difference in wavelength between a fluorochrome's excitation and emission is termed its Stokes shift after its discoverer.

If a fluorescent dye can be made to interact with specific cellular components—attached to an antibody that binds to a cellular protein, for example—then it can be used as a probe for microscopy. A specimen stained with this probe may be illuminated with pure, filtered light corresponding to its excitation wavelength and then viewed through an emission filter which is opaque to all other light except for its emission wavelength. The structures tagged with the fluorescent probe will appear to light up against a black background in a high contrast image.

The advent of fluorescence microscopy has given rise to the development of a number of Fluorescent dyes or reagents to selectively highlight in vivo the biological targets, biomarkers and pathways that underlie disease progression and therapeutic response. These agents enable in vivo imaging of biological processes. Non-fluorescent (optically silent) in their native (quenched) state, they generate high levels of fluorescence through enzyme-mediated release of their fluorochrome. An example of the use of such fluorescent dyes as biomarkers in diagnostic analysis is marketed by VisEn Medical Inc., Bedford, Mass. They have pioneered fluorescence-based Quantitative Tomography for in vivo imaging research. Imaging systems based on VisEn's proprietary FMT technology provide non-invasive, whole body, deep tissue imaging and generate 3D Images of the anatomy. These systems are used for research in oncology as well as inflammatory, pulmonary, cardiovascular and skeletal disease. Biological targets and pathways can be monitored and quantified in real time—giving a deeper understanding of the biology underlying disease mechanisms and therapeutic response. This technology overcomes some of the disadvantages associated with direct optical imaging. One of the key limitations of optical imaging is the natural scattering of photons by biological tissue. As a result of this scattering, there is no linearity between raw camera counts captured at the surface of an imaging subject and the true signal intensity emanating from within the subject, regardless of system calibration. Consequently, the depth, size and associated absolute fluorescence of a fluorescent signal cannot be accurately determined by camera count readouts from conventional imaging systems. Having administered the fluorescent reagent to the desired site, the technology uses Raster Scan Laser Light technology to measure absorption profiles and generates paired absorption and fluorescence data maps. All paired absorption and fluorescence data acquired is processed to generate normalized fluorescence measurements. Fluorescence quantification at each point in the subject and generated fluorescence measurements throughout regions of interest are calculated.

A number of other imaging technologies also exist and are becoming prevalent in the field including spectroscopy, light scattering spectroscopy, confocal microscopy, and cystoscopy. Optical Coherence Tomography, or ‘OCT’, is a technique for obtaining sub-surface images of translucent or opaque materials at a resolution equivalent to a low-power microscope. It is effectively ‘optical ultrasound’, imaging reflections from within tissue to provide cross-sectional images.

OCT is attracting interest among the medical community, because it provides tissue morphology imagery at much higher resolution (better than 10 μm) than other imaging modalities such as MRI or ultrasound.

The key benefits of OCT include but are not limited to: live sub-surface images at near-microscopic resolution, instant, direct imaging of tissue morphology, no preparation of the sample or subject, and no ionizing radiation.

OCT delivers high resolution because it is based on light, rather than sound or radio frequency. An optical beam is directed at the tissue, and a small portion of this light that reflects from sub-surface features is collected. Note that most light is not reflected but, rather, scatters. The scattered light has lost its original direction and does not contribute to forming an image but rather contributes to glare. The glare of scattered light causes optically scattering materials (e.g., biological tissue, candle wax, or certain plastics) to appear opaque or translucent even while they do not strongly absorb light (as can be ascertained through a simple experiment—e.g., shining a red laser pointer through one's finger). Using the OCT technique, scattered light can be filtered out, completely removing the glare. Even the very tiny proportion of reflected light that is not scattered can then be detected and used to form the image in, e.g., a scanning OCT system employing a microscope.

The physics principle allowing the filtering of scattered light is optical coherence. Only the reflected (non-scattered) light is coherent (i.e., retains the optical phase that causes light rays to propagate in one or another direction). In the OCT instrument, an optical interferometer is used in such a manner as to detect only coherent light. Essentially, the interferometer strips off scattered light from the reflected light needed to generate an image. In the process depth and intensity of light reflected from a sub-surface feature is obtained. A three-dimensional image can be built up by scanning, as in a sonar or radar system.

The need for accurate and precise measurements of organs, tissues, structures, and sub-structures continues to increase. For example, in following the response of a disease to a new therapy, the accurate representation of three-dimensional (3D) structures is vital in broad areas such as neurology, oncology, orthopedics, and urology. In human and animal anatomy texts, there are a great number of named organs, structures, and sub-structures. Furthermore, in disease states modifications to normal structures are possible and additional pathological structures or lesions can be present. Despite the imposing number of defined sub-structures and pathologies, within the major disease categories there are specific objects that serve as indicators of disease. For example, liver metastases, brain lesions, atherosclerotic plaques, and meniscal tears are some examples of specific indicators of different conditions. The topological, morphological, radiological, and pharmacokinetic characteristics of biological structures and sub-structures are called biomarkers, and specific measurements of the biomarkers can provide a quantitative assessment of disease progress. The ability to clearly and precisely quantify, distinguish and identify these biomarkers represents a needed and important step for an accurate, image-based assessment of both normal and disease states.

### Delivery of Optical Imaging Capabilities

The invention described herewith provides additional embodiments of the present invention to facilitate the delivery of optical imaging capability to areas in the GI and respiratory tracts as well as the abdominal cavity.

EUS and EBUS guided FNA as mentioned previously, are two procedures whereby ultrasound can be used to guide a needle device to an intended anatomical site to extract tissue samples, fluid, cells etc., from suspicious masses in the GI and Respiratory tracts. A challenge in the exploration and treatment of internal areas of the human anatomy has been adequately visualizing the area of concern. Visualization can be especially troublesome in minimally invasive procedures in which small diameter, elongate instruments, such as catheters or endoscopes, are navigated through natural passageways of a patient to an area of concern either in the passageway or in an organ reachable through the passageway.

Once EUS has been performed, the patient may be referred for a follow up endoscopic procedure such as ERCP to further evaluate disease in the GI tract including the biliary and pancreatic ductal areas. In such procedures, therapy may be directed to sites in the biliary and pancreatic ductal regions under direct visualization via the use of a miniature endoscope or fiber optic probe (see e.g., US Patent Application 2005/0272975 A1 and US Patent Application 2006/0264919 A1, the contents of which are herein incorporated by reference in their entireties).

The need for a second procedure to directly visualize desired anatomical areas, as distinct from the original EUS procedure, has the disadvantages of providing additional clinical risk to the patient, increased procedural costs to both patient and hospital institutions, and poor procedural efficiency.

Furthermore, the ability to cannulate the hepatic ducts, the common hepatic duct, the common bile duct, the cystic duct and the pancreatic ductal systems through the wall of the stomach in conjunction with EUS guided FNA, provides the physician with a “tract” or pathway for the advancement of catheters, stent delivery systems, Electro Hydraulic Lithotripsy (EHL) probes, Laser ablation fibers, cholangioscopes, ultrasound probes, confocal imaging probes etc. . . . into these areas. In this instance, a guidewire may be passed through the needle housing member component into the desired area of choice, providing a track for the passage of the aforementioned devices.

As such, there is a clinical need to be able to provide the endoscopist or pulmonologist with a “real—time” ability to visualize areas of the GI and tracheo-bronchial tracts in conjunction with EUS and/or EBUS FNA and to, if desired, direct therapy to these areas, as part of the same procedure.

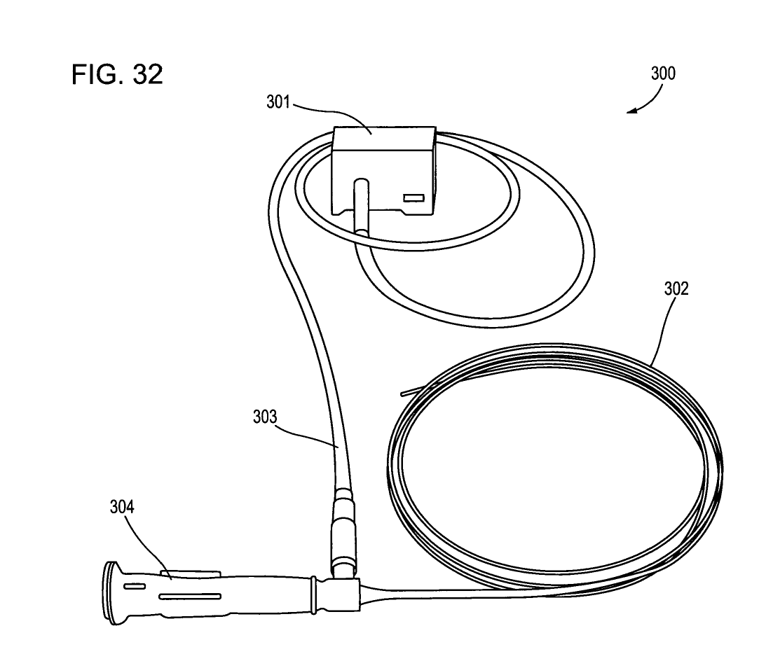

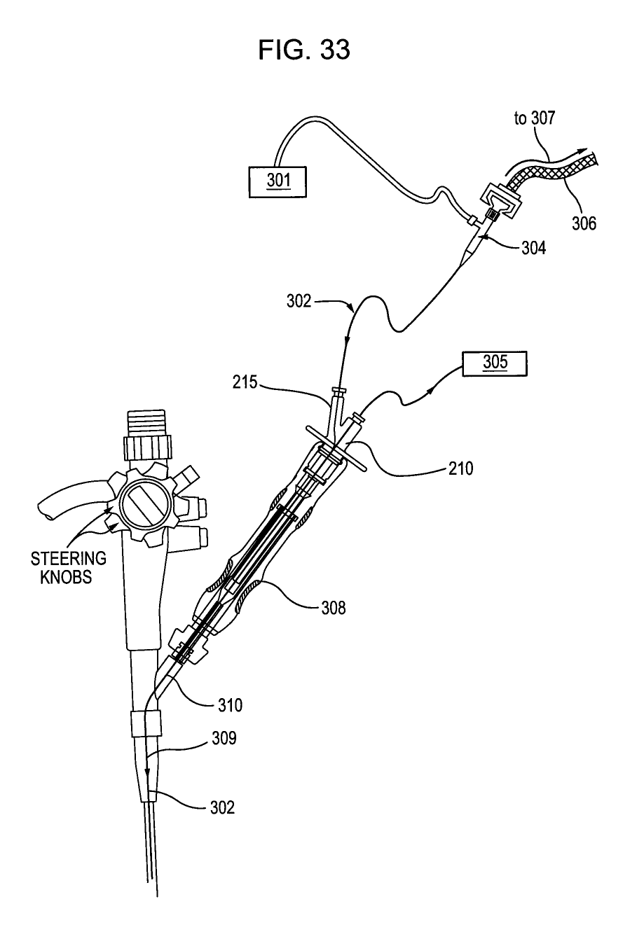

The FNA catheter system illustrated in FIG. 17 may be used in conjunction with a fiber optic imaging system to directly visualize areas of GI and tracheo-bronchial tracts or surrounding areas in the abdominal cavity. FIG. 32 depicts a typical fiber optic imaging system 300, which is known to persons skilled in the art of diagnostic endoscopy. The fiber optic imaging system shown in FIG. 32 includes a light source 301, a fiber optic catheter 302, a fiber optic cable 303, and an occular 304. FIG. 33 is an assembly drawing of how the fiber optic imaging system 300 may be used in conjunction with EUS or EBUS FNA. The FNA catheter 308 is attached to the echoendoscope 309 through a working channel port 310, as previously described. In the event that direct visualization is desired for further evaluation, the fiber optic probe 302 may be inserted down the bifurcated side port 215 in the bifurcated hub of the needle housing member 205 to exit distally at the bevel end of the needle component, in vivo. Alternately, the needle housing member may be removed from the catheter and fiber optic probe component loaded through the catheter handle/catheter sheath to exit distally in vivo. The primary port 210 of the hub on the needle housing member is attached to a peristaltic (or other) pump 305 capable to delivering saline and/or de-ionized water to the distal end of the needle. This liquid media injection is required to ensure that the field of view being visualized remains clear and that the image is more readily discernable. The fiber optic probe 302 which houses both fiber optic image fibers (which transmit the image) and illumination fiber bundles (which are routed to the illumination cable providing light) is housed in an adjustable occular 304 at the distal end. This occular 304 magnifies the transmitted image from the fiber optic cable 303. A CCD (charge-coupled device) or CMOS (complementary metal-oxide-semiconductor) camera 306 processes the image and transmits the image to a camera system (screen) 307 thus providing real time image analysis of intended sites. The fiber optic probe 302 may be advanced and retracted through the needle housing member component of the embodiment as desired.

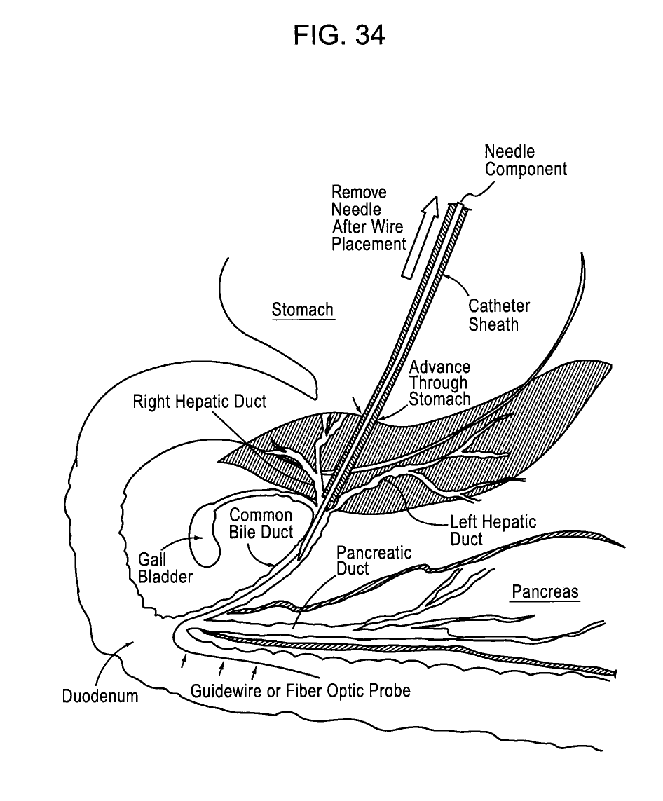

FIG. 34 illustrates how the current invention may be used in conjunction with a fiber optic imaging system to advance the FNA catheter and needle housing member across the stomach wall to cannulate ducts in the biliary system (for example the left and right hepatic ducts, the common bile duct, the cystic duct etc.) and to conduct direct visualization of these ductal systems. Depending upon the outcome of visual inspection of the biliary tree, the endoscopist may decide at this stage to perform a therapeutic procedure in the biliary system. In this instance, the FNA catheter and needle housing member may be used to provide the physician with a track to exchange or advance ancillary devices through the stomach wall, trans-ductally (or trans-hepatically). In this instance, depth of the needle is locked in position as previously described. The fiber optic probe is removed from the needle housing member. A guidewire (which may range in size from 0.010″ diameter to 0.038″ diameter) is advanced through the needle housing member from a proximal position, ex-vivo. The guidewire is advanced to extend through the distal end of the needle and tracked through the common bile duct to reside across the papilla or alternately, may be advanced down the intra-hepatic branches. The needle housing member and/or FNA catheter may then be disconnected from the echoendoscope leaving the guide wire to provide a track trans-orally. Thereafter, ancillary devices such as catheters, stent delivery systems, cholangioscopes, EHL devices, stone retrieval devices etc., may be advanced over the wire into the biliary tree for evaluation.

In addition to using the current FNA catheter and exchangeable needle housing member device in conjunction with fiber optic probe delivery, the FNA catheter and exchangeable needle housing member may also be used to deliver confocal imaging probes to desired sites within the GI and respiratory tracts to aid in the diagnosis of biliary and tracheo-bronchial abnormalities.

In a conventional epifluorescence imaging probe, short wavelength light (e.g. blue light) is reflected by a chromatic reflector through the objective and bathes the whole of the specimen in uniform illumination. The chromatic reflector has the property of reflecting short wavelength light and transmitting longer wavelength light. Emitted fluorescent light (e.g. longer wavelength, green light) from the specimen passes straight through the chromatic reflector to the eyepiece (occular when referring to FIG. 33).

In a confocal imaging system a single point of excitation light (or sometimes a group of points or a slit) is scanned across the specimen. The point is a diffraction limited spot on the specimen and is produced either by imaging an illuminated aperture situated in a conjugate focal plane to the specimen or, more usually, by focusing a parallel laser beam. With only a single point illuminated, the illumination intensity rapidly falls off above and below the plane of focus as the beam converges and diverges, thus reducing excitation of fluorescence for interfering objects situated out of the focal plane being examined. Fluorescent light (i.e. signal) passes back through a dichroic reflector and then passes through a pinhole aperture situated in a conjugate focal plane to the specimen. Any light emanating from regions away from the vicinity of the illuminated point will be blocked by the aperture, thus providing yet further attenuation of out-of focus interference. Light passing through the image pinhole is detected by a photodetector. Usually a computer is used to control the sequential scanning of the sample and to assemble the image for display onto the video monitor. Most confocal imaging probes are implemented as imaging systems that couple to a conventional receiver.

In addition to using the current FNA catheter and exchangeable needle housing member device in conjunction with fiber optic probe delivery, the FNA catheter and exchangeable needle housing member, in a further embodiment, may also be used to deliver ultrasound probes to desired sites within the GI and respiratory tracts to aid in the diagnosis of biliary and tracheo-bronchial abnormalities.

Ultrasound probes have been broadly used in the GI and tracheo-bronchial fields to examine abnormalities in these areas. Typical ultrasound probes used in the medical field have been described, for example, by Ishiguro et al, U.S. Pat. No. 5,131,393 and Tanaka et al, U.S. Pat. No. 5,368,036 (the contents of which are herein incorporated by reference in their entireties) and are known to persons skilled in the art of diagnostic ultrasonography. In the event that direct visualization is desired for further evaluation, as it relates to the present invention, the ultrasound probe may be inserted down the bifurcated side port in the hub of the needle housing member to exit distally at the bevel end of the needle component, in vivo. (Alternately, the needle housing member may be removed from the catheter and fiber optic probe component loaded through the catheter handle/catheter sheath to exit distally in vivo). The ultrasound probe usually incorporates a piezoelectric transducer encased in the distal end of the probe. Strong, short electrical pulses from the ultrasound machine make the transducer ring at the desired frequency. The frequencies can be anywhere between 2 and 18 MHz. The sound is focused either by the shape of the transducer, a lens in front of the transducer, or a complex set of control pulses from the ultrasound scanner machine. This focusing produces an arc-shaped sound wave from the face of the transducer. The wave travels into the body and comes into focus at a desired depth.

More modern ultrasound technology transducers use phased array techniques to enable the sonographic machine to change the direction and depth of focus. Materials on the face of the transducer enable the sound to be transmitted efficiently into the body (usually seeming to be a rubbery coating, a form of impedance matching). In a similar fashion, the exchangeable needle housing member of the present invention may be used to deliver a range a probes (enabling OCT, confocal microscopy, fluorescence marker imaging etc.) to various bodily areas to supplement conventional 2-D Ultrasound images.

The sound wave is partially reflected from the layers between different tissues. Specifically, sound is reflected anywhere there are density changes in the body: e.g. blood cells in blood plasma, small structures in organs, etc. Some of the reflections return to the transducer. The return of the sound wave to the transducer results in the same process that it took to send the sound wave, except in reverse. The return sound wave vibrates the transducer the transducer turns the vibrations into electrical pulses that travel to the ultrasonic scanner where they are processed and transformed into a digital image.

### Delivery of Self-Expanding Stents

In a further embodiment of the invention disclosed herewith, the needle housing member facilitates the delivery of stent, such as a metal or polymeric self expanding or laser cut stent, which may be used as a conduit to provide drainage in the gastrointestinal system between the following anatomical entities, including the gall bladder and the left hepatic and stomach, gall bladder drainage from the gall bladder to the duodenum, gall bladder drainage from the gall bladder to the stomach, the pancreas to the stomach and uncinate of the duodenum, and pseudocyst drainage into the stomach.

Self-expanding medical prostheses frequently referred to as stents are well known and commercially available. Devices of these types are used within body vessels of humans and other animals for a variety of medical applications. Examples include intravascular stents for treating stenoses, stents for maintaining openings in the urinary, biliary, esophageal and renal tracts and vena cava filters to counter emboli. Briefly, self-expanding stents of the type described in the above-identified patent documents are formed from a number of resilient filaments which are helically wound and interwoven in a braided configuration. The stents assume a substantially tubular form in their unloaded or expanded state when they are not subjected to external forces. When subjected to inwardly directed radial forces the stents are forced into a reduced-radius and extended-length loaded or compressed state. A delivery device which retains the stent in its compressed state is used to deliver the stent to a treatment site through vessels in the body. The flexible nature and reduced radius of the compressed stent enables it to be delivered through relatively small and curved vessels. After the stent is positioned at the treatment site the delivery device is actuated to release the stent, thereby allowing the-stent to self-expand within the body vessel. The delivery device is then detached from the stent and removed from the patient.

Cholecystitis is often caused by cholelithiasis (the presence of choleliths, or gallstones, in the gallbladder), with choleliths most commonly blocking the cystic duct directly. This leads to inspissation (thickening) of bile, bile stasis, and secondary infection by gut organisms, predominantly E. coli and Bacteroides species. In typical cases, the gallbladder's wall becomes inflamed. Extreme cases may result in necrosis and rupture. Inflammation often spreads to its outer covering, thus irritating surrounding structures such as the diaphragm and bowel. Less commonly, in debilitated and trauma patients, the gallbladder may become inflamed and infected in the absence of cholelithiasis, and is known as acute acalculous cholecystitis. The patient might develop a chronic, low-level inflammation which leads to a chronic cholecystitis, where the gallbladder is fibrotic and calcified. In such instances where the cystic duct has become blocked and cannot be curatively treated via lithotripsy to break up cystic duct stones or the placement of a stent, trans-papillary, the patient may undergo a laproscopic cholecystectomy, whereby the gallbladder is removed laproscopically and the cystic duct is tied off. This type of procedure causes obvious trauma to the patient and requires a high level of skill on the part of the physician. The placement of a stent or prostheses transgastrically however, which would provide drainage from the gall-bladder to the stomach or duodenum may alleviate these concerns and provide for a more efficient treatment algorithm compared to laproscopic cholecystectomy in the treatment of cholecystitis.

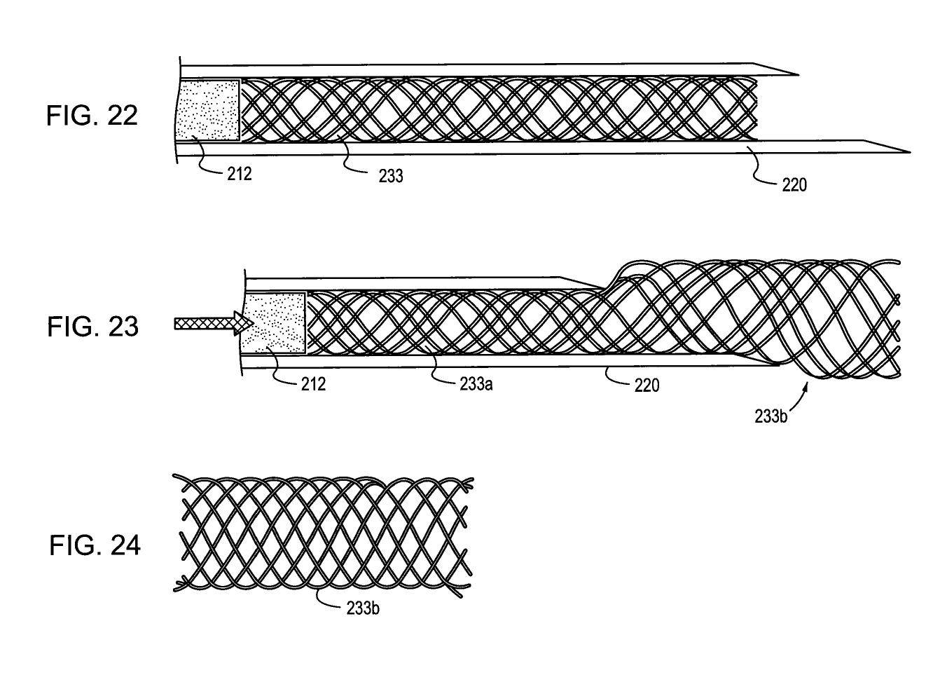

The needle and/or catheter sheath of the device is loaded with a self expanding metal or polymeric stent 233 which can be used as a conduit between various organs in the GI and tracheo-bronchial systems. Illustrations of the embodiment(s) are shown in FIGS. 22 through 27 inclusive.

FIGS. 22 through 24 inclusive show embodiments if the distal end of the needle component of the device with a self expanding stent encapsulated inside the distal needle end. The stent 233 in FIG. 22 is shown in it's compressed state. The stent is deformed in the longitudinal direction and is supplied, pre-loaded to the user, in the body of the needle component 220 at the distal end of same. If the physician so desires to place a stent 233 to act as a conduit between various bodily organs described above, the needle component 220 of the needle housing member with pre-loaded stent 233 is loaded into the proximal end of the FNA catheter and securely locked to the handle aspect of the FNA Catheter. The needle is advanced to the desired anatomical site. Once the needle is positioned appropriately under endoscopic ultrasound or fluoroscopic guidance, the stylet 212 is loaded into the primary port (sometimes referred to herein as a luer port) external to the patient subject. The stylet is then advanced to push and deploy the stent into the desired location as shown in FIG. 23, such that the stent self-expands from a compressed state 233a to an expanded state 233b once deployed form the needle. FIG. 24 is a drawing depicting the stent 233b post expansion in the free state, ex-vivo. In this way, the bevel of the needle serves to penetrate through the tumor mass or bodily organ under treatment and provides a path for deployment of the stent.

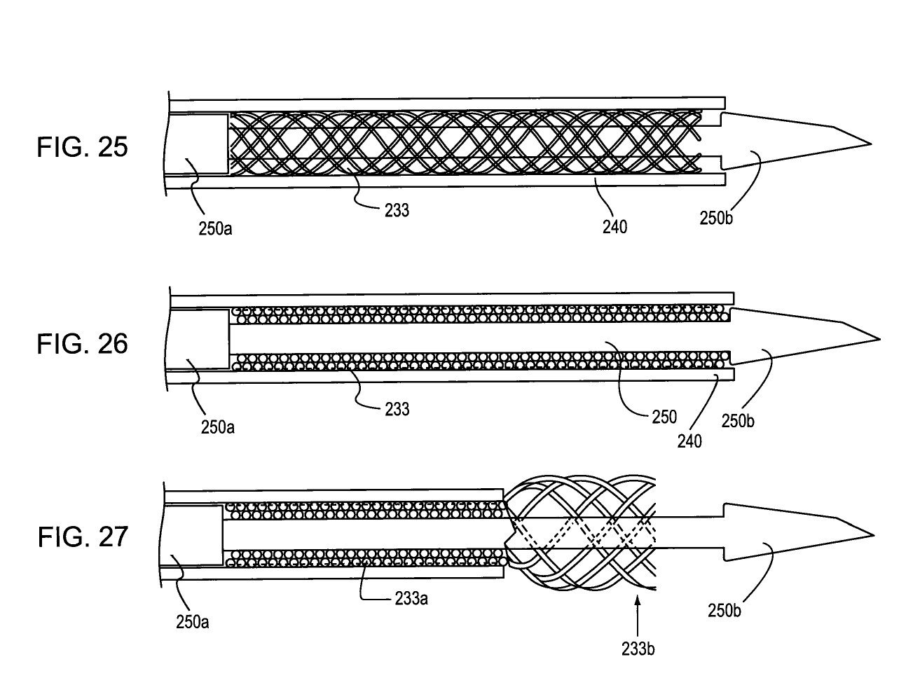

FIGS. 25-27 depict an alternative embodiment of a self-expanding stent pre-loaded onto a distal portion of the needle component of the needle housing member. Instead of being pre-loaded within the lumen of the needle, as depicted in FIG. 25, the self-expanding stent is loaded onto the needle between a proximal portion of the needle 250a and a needle trocar 250b, (collectively needle 250) such that the self-expanded stent is disposed between the catheter sheath 240 and the needle/trocar 250a/250b. The needle/trocar 250a/250b may be recessed to house the stent. As the needle is deployed from the catheter sheath 240, the stent self-expands from the compressed state 233a to the expanded state 233b.

As before stated, self expanding stents such as those described herein are commonly used in the fields of esophageal, biliary and tracheo-bronchial stenting (see e.g., U.S. Pat. No. 5,888,201, the contents of which are herein incorporated by reference in its entirety). However, these stents are typically delivered in a secondary medical procedure which causes additional expense to the patient and institution. Self expanding stents, in the present disclosure, may be delivered in conjunction with FNA, thus combining a diagnostic and therapeutic procedure into a single procedure and thus reducing the amount of trauma to the patient and increasing the efficiency of therapeutic administration. As before stated, the invention disclosed herewith, may be used to deliver a self expanding stent to “bridge” between the following anatomical GI landmarks: the left hepatic and stomach, gall bladder drainage from the gall bladder to the duodenum, gall bladder drainage from the gall bladder to the stomach, gall bladder drainage from the gall bladder to the duodenum, the pancreas to the stomach and uncinate of the duodenum, and pseudocyst drainage into the stomach.

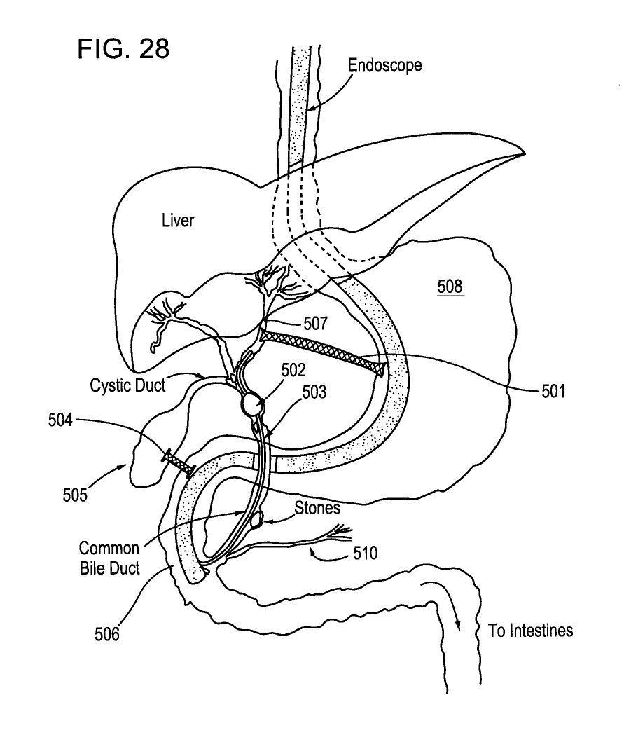

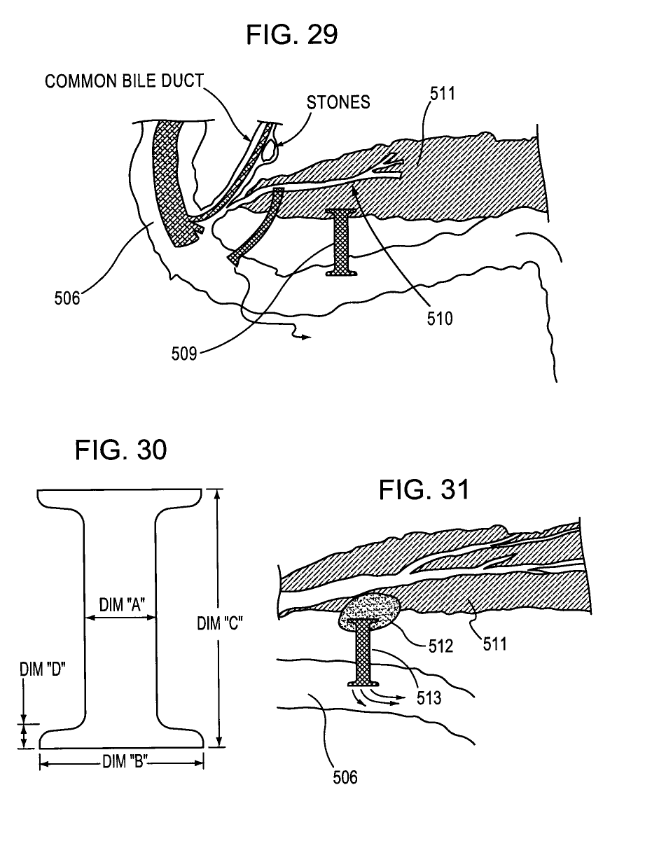

FIGS. 28 and 29 are illustrative of stenting of some of the typical areas identified above. In the case of stenting to provide drainage for pseudocysts, the pancreatic pseudocyst, the most common cystic lesion of the pancreas, is a localized collection of fluid rich in amylase within or adjacent to the pancreas and enclosed by a non-epithelialized wall, occurring as a result of acute or chronic pancreatitis, pancreatic trauma, or pancreatic duct obstruction. As shown in FIG. 28, a stent (e.g., self-expanding stent) 501 may be placed between the left hepatic 507 and stomach 508 using the needle biopsy devices described herein. A stent (e.g., a balloon wire) 502 may also be placed in a duct 503. A stent 504 can also be placed between the gall bladder 505 and duodenum 506. As shown in FIG. 73, a stent 509 can deployed between the pancreatic duct 510 and duodenum 506 using the devices described herein. A stent can also be deployed between the pancreas 511 and duodenum 506.

Currently, at least 3 major forms of therapy are available: percutaneous drainage, surgical intervention, and endoscopic drainage. Controversy exists concerning which of these techniques should be offered to the patient as initial therapy. Three options exist for the surgical management of pancreatic pseudocysts: excision, external drainage, and internal drainage. Surgery, which traditionally was the major treatment approach for pancreatic pseudocysts, has been challenged by newer endoscopic techniques. Given the low complication and mortality rates and the high success rate of endoscopic drainage when compared with surgery, surgical intervention should be reserved only for certain cases. The addition of endoscopic ultrasonography (EUS) for endoscopic drainage is a new and exciting development and may decrease the risks associated with endoscopic drainage. There are many complications with the aforementioned methods of pseudocyst treatment (for example, significant contamination of the abdominal cavity, free perforation of the stomach, and hemorrhage.) Thus the embodiment disclosed herewith of stent delivery for pseudocyst drainage incorporation into and used in conjunction with EUS guided FNA offers many obvious advantages of in terms of patient safety and clinical efficiency.

FIGS. 25 through 27 are representative of a preferred alternate design to facilitate stent expanding stent delivery in conjunction with a EUS or EBUS FNA device. In this instance, the needle component may be hollow (hypo-tube) in nature or as illustrated in FIG. 25, the needle be a solid member incorporating a beveled trocar detail at this distal end. Referring to FIGS. 26 and 27, the needle element incorporates a stepped transition at the distal end. The stent is loaded onto the exterior recess portion of the needle/trocar at the distal end. The catheter sheath is then advanced over the needle/trocar and compressed stent such that the stent remains compressed and unexpanded to facilitate delivery. As before if the physician desires to place a stent to act as a conduit between various bodily organs as described above, the needle housing member with pre-loaded stent in the needle component thereof is loaded into the proximal end of the FNA catheter and securely locked to the handle aspect of the FNA Catheter. The needle is advanced to the desired anatomical site. Once the needle is positioned appropriately under endoscopic ultrasound or fluoroscopic guidance, the catheter sheath member is retracted (or alternately, the needle/trocar element is advanced) which allows the stent to expand due to the removal of the external constricting force of the sheath.

A potential stent design to facilitate pseudocyst drainage into the stomach is presented in FIG. 30. Dim “A” (stent body diameter may vary from 2-20 mm but is most preferably of the order of 2-8 mm in diameter); Dim “B” (stent flare diameter may vary from 2-20 mm but is most preferably of the order of 2-12 mm in diameter); Dim “C” (stent length may vary from 5-180 mm but is most preferably of the order of 10-80 mm in length); Dim “D” (stent flare length may vary from 2-20 mm but is most preferably of the order of 2-4 mm).

FIG. 31 illustrates the typical location for placement of a stent 513 to facilitate drainage of a pancreatic pseudocyst 512 into the duodenum 506.

In the case of self expanding stents such as those disclosed herewith, commonly used materials for the stent filaments include Elgiloy® and Phynox® spring alloys. Both of these metals are cobalt-based alloys which also include chromium, iron, nickel and molybdenum. Other materials used for self-expanding stent filaments are stainless steel and MP35N alloy and superelastic Nitinol nickel-titanium alloy which contains approximately 45% titanium. Elgiloy®, Phynox®, MP35N and stainless steel are all high strength and high modulus metals. Nitinol® has a relatively lower strength and modulus. There remains a continuing need for self-expanding stents with particular characteristics for use in various medical indications. Stents are needed for implantation in an ever growing list of vessels in the body. Different physiological environments are encountered and it is recognized that there is no universally acceptable set of stent characteristics. In particular, there is a need for stents formed from moderate strength materials having lower moduli of elasticity than those of Elgiloy®, Phynox®, MP35N, and stainless steel from which certain stents are currently formed. Stents formed from moderate strength and relatively low moduli of elasticity materials would have properties adapted to an expanded range of treatment applications. Stents with lower moduli of elasticity material would be less stiff and more flexible than a stent made of the same size wire and same design utilizing a high modulus material. Stents of these types must also exhibit a high degree of biocompatibility. Furthermore, the filaments from which the stent is fabricated are preferably radiopaque to facilitate their implantation into patients. The stent will preferably be capable of withstanding radially occlusive pressure from tumors, plaque, and luminal recoil and remodeling.

### Real-Time Validation of Aspirated Cellular Sample

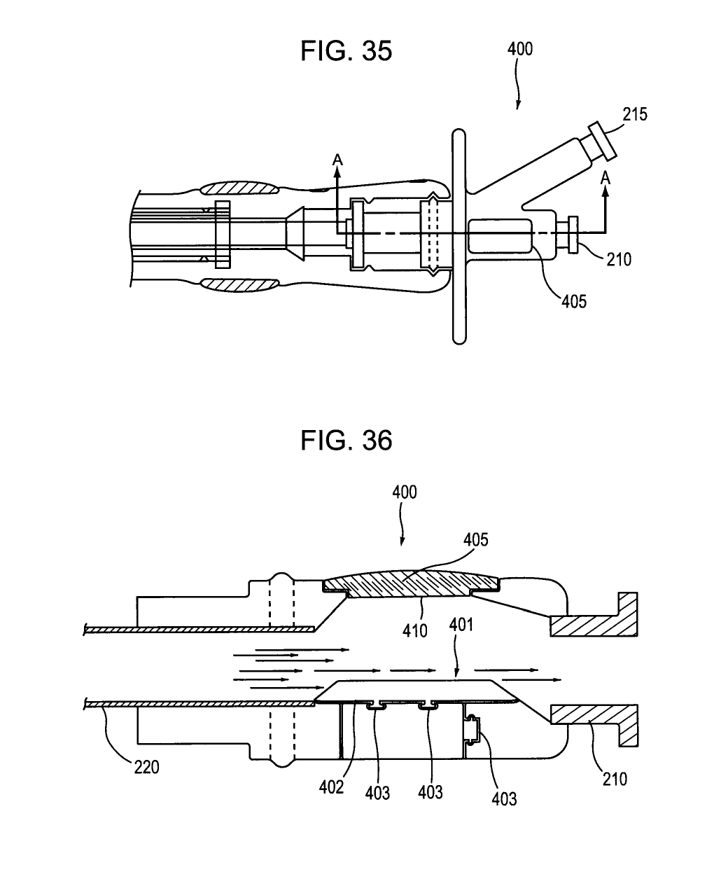

An alternate embodiment for an exchangeable needle housing member is presented in FIGS. 35 and 36 respectively. FIG. 36 is an expanded cross-sectional view of the portion of FIG. 35 in-between the arrows designated as “A”. In embodiment, the hub 400 of the needle housing member design incorporates the ability to provide the end user with real-time validation of the aspirated cellular sample. Referring to FIG. 36, the needle hub 400 contains an assay loaded insert indicator 401 housed internally and mounted to the inner wall 402 of the needle hub. The assay loaded insert indicator 401 is manufactured from a material capable which has been loaded, imbibed or compounded with an assay reagent. Such materials may consist of polymers such as polyurethane, polyethylene, cellulose acetate, polyvinyl-alcohol, elastomers or oligomers or copolymers thereof. Immediately perpendicular to the assay loaded insert indicator 401, a clear window aperture component 405 is mounted externally on the opposing wall 410 of the needle hub body 400. This clear window aperture component 405 may be manufactured from transparent/translucent material such as glass or thermoplastic polymer materials such as polyamide, poly-methyl-methacrylate, polycarbonate, polystyrene or derivatives thereof. The clear window aperture component may be attached to the needle hub body via adhesive bonding or via a range of snap-fit mechanical fastening techniques. Similarly, the assay loaded insert indicator 401 may be attached to the inner surface 402 of the needle hub body 400 via adhesive bonding or via a range of snap-fit mechanical fastening techniques 403.

As shown in FIG. 36, the sample is aspirated for the anatomical site per conventional FNA procedure, and the aspirated sample flows through the needle 220 in the direction of the arrows. As the cellular sample is retracted to contact the assay loaded insert indicator, the cellular sample reacts with the assay in the assay loaded insert indicator component 401, which results in the assay loaded insert indicator changing color. This color changed may then be externally viewed by the user through the clear window aperture 405. It is preferable that the assay loaded insert indicator 401 change color to a specific color denoting the presence of benign cells in the sample and change to an alternate color denoting the presence of malignant sample cells. In this way, a real time, positive/negative diagnosis can be made by the endoscopist or pulmonologist alone, or in conjunction with the cystopathologist.

### Delivery of Neuromodulation and Pacing Leads

The present invention described herewith may also be used to deliver neuromodulation/pacing leads to specific areas in the GI and respiratory tracts as well as other areas of the human anatomy.

Neuromodulation is the alteration of nerve activity through the delivery of electrical stimulation or chemical agents to targeted sites of the body. Neuromodulation works by either actively stimulating nerves to produce a natural biological response or by applying targeted pharmaceutical agents in tiny doses directly to site of action.

Alternatively, minute pacing leads may be delivered to specific areas in the body to stimulate sensory responses. These precisely placed leads connect via an extension cable to a pulse generator and power source, which generates the necessary electrical stimulation. A low-voltage electrical current passes from the generator to the nerve, and can either inhibit pain signals or stimulate neural impulses where they were previously absent.

In the case of pharmacological agents delivered through implanted pumps, the drug can be administered in smaller doses because it does not have to be metabolized and pass through the body before reaching the target area. Smaller doses—in the range of 1/300 of an oral dose—can mean fewer side effects, increased patient comfort and improved quality of life.

In combination with the present invention, neuromodulation leads (pacing or other) may be delivered through the needle aspect of the invention and attached to nerve endings close to various GI or respiratory organs in the peripheral nervous system. In this way, functional motility of specific organs can be examined and modified as required. For example, neuromodulation leads may be attached to the wall of the stomach for GI pacing. In another example, pacing leads may be attached to the diaphragm so that the diaphragm may be paced. In the latter case, the placement of such leads on the diaphragm may be advantageous in helping to remove patient subjects from a ventilator via the administration of electrical impulses to control and regulate breathing.

Certain embodiments according to the invention have been disclosed. These embodiments are illustrative of, and not limiting on, the invention. Other embodiments, as well as various modifications and combinations of the disclosed embodiments, are possible and within the scope of the disclosure.

What is claimed is:

1. A device for needle biopsy, comprising:
a handle member having proximal and distal portions;
a proximal handle member disposed to the proximal portion of the handle member;
a distal handle member disposed to the distal portion of the handle member;
a sheath lumen disposed within the handle member and extending from the distal portion of the handle member;
a needle housing member partially disposed in the proximal handle member and comprising at least two ports for introducing a device or agent, wherein said needle housing member is moveable in a substantially transverse direction relative to the longitudinal axis of the handle member; and
a needle disposed within the sheath lumen.

2. The device of claim 1, wherein said agent is a therapeutic agent selected from the group consisting of: a chemotherapeutic agent, a sclerosing agent, a necrosing agent, a growth factor, and a radiation agent.

3. The device of claim 1, wherein said device introduced into the needle housing member is a stylet, a syringe or a fiber optic probe.

4. The device of claim 1, wherein the needle housing member further comprises a strain relief.

5. The device of claim 1, wherein the needle housing member further comprises a connecting member comprising at least one indentation for engaging to at least one adaptation member in the proximal handle member.

6. The device of claim 1, wherein the proximal handle member includes a release member that engages and disengages the needle housing member.

7. The device of claim 6, wherein the release member is depressible.

8. A device for needle biopsy, comprising:
a handle member having proximal and distal portions;
a proximal handle member disposed to the proximal portion of the handle member;
a distal handle member disposed to the distal portion of the handle member;
a sheath lumen disposed within the handle member and extending from the distal portion of the handle member;
a needle housing member partially disposed in the proximal handle member, wherein said needle housing member is moveable in a substantially transverse direction relative to the longitudinal axis of the handle member; and
a needle disposed within the sheath lumen, said needle comprising an agent or a device disposed at a distal portion thereof.

9. The device of claim 8, wherein said agent is a therapeutic agent selected from the group consisting of: a chemotherapeutic agent, a sclerosing agent, a necrosing agent, a growth factor, and a radiation agent.

10. The device of claim 8, wherein said agent is a diagnostic agent selected from the group consisting of a fiduciary marker, a biomarker, and an imaging probe.

11. The device of claim 8, wherein said agent is encapsulated in a membrane or capsule.

12. The device of claim 8, wherein said agent comprises a pellet or a seed form.

13. The device of claim 8, wherein said device disposed at the distal portion of the needle is a pacing lead or a stent.

14. The device of claim 8, wherein said needle housing member further comprises at least one port for introducing a device.

15. The device of claim 14, wherein said device introduced into the needle housing member is a stylet, a syringe or a fiber optic probe.

16. The device of claim 8, wherein the needle housing member further comprises a connecting member comprising at least one indentation for engaging to at least one adaptation member in the proximal handle member.

17. The device of claim 16, wherein the proximal handle member includes a release member that engages and disengages the needle housing member.

18. The device of claim 17, wherein the release member is depressible.

19. A device for needle biopsy, comprising:
a handle member having proximal and distal portions;
a proximal handle member disposed to the proximal portion of the handle member;
a distal handle member disposed to the distal portion of the handle member;
a sheath lumen disposed within the handle member and extending from the distal portion of the handle member;
a needle housing member partially disposed in the proximal handle member and comprising an indicator assay insert mounted to an inner wall in a proximal portion of the needle housing member
needle hub contains an assay loaded insert indicator housed internally and mounted to the inner wall of the needle hub; and
a needle disposed within the sheath lumen.

20. The device of claim 19, wherein said needle housing member further comprises a clear window in the proximal portion of the needle housing member.
